(19) United States
(12) Patent Application Publication
(10) Pub. No.: US 2026/0308525 A1
(43) Pub. Date: Oct. 8, 2026

(54) METHODS OF ENHANCING OR MODIFYING NK CELLS

(71) Applicant: ZELLUNA IMMUNOTHERAPY AS, Oslo (NO)

(72) Inventors: Emilie GAUTHY, Oslo (NO); Giulia MALACHIN, Oslo (NO); Julia INO, Oslo (NO); Pranav OBEROI, Oslo (NO); Artur CIESLAR-POBUDA, Oslo (NO)

(21) Appl. No.: 19/492,110

(22) PCT Filed: Jun. 19, 2024
(86) PCT No.: PCT/EP2024/067073

§ 371 Date: Dec. 10, 2025

(30) Foreign Application Priority Data
Jun. 20, 2023 (GB) 2309290.1

(51) Int. Cl. C12N 5/0783; A61K 40/15; A61K 40/32; C07K 14/705; C12N 15/86

(52) U.S. Cl. CPC C12N 5/0646; A61K 40/15; A61K 40/32; C07K 14/7051; C12N 15/86; C12N 2501/2302; C12N 2501/2315; C12N 2501/515; C12N 2502/30; C12N 2510/00; C12N 2740/15043

(57) ABSTRACT

The present invention relates to methods of enriching, enhancing, modifying, and/or generating populations of natural killer (NK) cells that are suitable for therapeutic uses. For example, the invention relates to populations of T cell receptor (TCR)-NK cells with therapeutic utility and to improved methods of making said cells. The invention also relates to methods of treatment comprising the use of said cells.

### FIELD OF THE INVENTION

The present invention relates to methods of enriching, enhancing, modifying, and/or generating populations of natural killer (NK) cells that are suitable for therapeutic uses. For example, the invention relates to populations of T cell receptor (TCR)-NK cells with therapeutic utility and to improved methods of making said cells. The invention also relates to methods of treatment comprising the use of said cells.

### BACKGROUND OF THE INVENTION

Immunotherapeutic approaches for the treatment of pathologies, including the treatment of viral diseases and of cancers, are proving increasingly efficacious. Some such therapies are based on the transfer of NK cells to patients.

NK cells have an innate activity that enables the targeting of cells that are associated with particular pathologies. In addition to this innate activity, it can be desirable to modify the NK cells to have additional functionalities and to boost their therapeutic potential.

Examples of desirable modifications include the modification of NK cells to express a functional TCR. This endows the NK cell with the potential for TCR-directed cytotoxicity and hence enables the use of NK cells to target cells expressing specific antigens. This approach is described in WO2016/116601A1. A later publication discussing the TCR-NK approach is WO2018/129199A1. NK cells that have been modified in this manner are referred to as “TCR-NK cells” herein.

In another example, NK cells may be modified to express a chimeric antigen receptor (CAR). For example, NK cells may be modified to express a CAR that comprises an antigen-binding fragment of a TCR. This approach is disclosed in WO2019/069125A1.

Thus, there is a need for processes to produce populations of NK cells that include exogenous nucleic acids, for instance nucleic acids that enable expression of TCRs, where the populations are suitable for therapeutic purposes. Desirable properties of such populations include: the proportion of the population that is an NK cell, the proportion of the NK cells that comprise exogenous genes, the viability of the cells, the performance of the cells in functional assays (such as killing assays), the overall yield of relevant cells, and similar properties.

The prior art discloses the successful transduction of NK cells using viral vectors. Within the prior art, there have been attempts to optimise the best conditions for the production of a transduced NK cell population.

U.S. Pat. No. 11,293,010 B2 reports methods of producing NK cells that include one or more heterologous nucleic acids. The authors report that transduction after 2 to 3 days of prior cytokine stimulation is preferred (U.S. Pat. No. 11,293,010 B2, FIG. 14).

Another publication that reports methods of transducing NK cells is Sutlu et al. (HUMAN GENE THERAPY 23:1090-1100 (October 2012)). FIG. 4 of this publication shows a plateau in transduction percentage at day 2 post stimulation.

Su et al. (Blood (2011) 118 (21): 4714) tested the efficiency of lentiviral transduction when NK cells were pre-stimulated in vitro for 24 hours in media containing IL-2 (200 U/mL) and when NK cells underwent in vitro expansion over 9 days prior to transduction using irradiated EBV-LCL feeder cells and media containing IL-2 (200 U/mL).

Childs and Berg (Hematology Am Soc Hematol Educ Program. 2013; 2013(1):234-46) review methods to activate and expand human NK cells ex vivo for adoptive transfer in humans.

Optimal conditions for activation, transduction, enrichment, and expansion remain to be determined.

### SUMMARY OF THE INVENTION

In an aspect of the present disclosure, there is provided a method of enriching natural killer (NK) cells that express a CD3-TCR complex (TCR-NK cells) within a population of cells, the method comprising:
obtaining a population of cells comprising TCR-NK cells; and
enriching for TCR-NK cells using surface CD3 as a marker.

In an aspect of the present disclosure, there is provided a method for enhancing one or more functions of a population of cells comprising TCR-NK cells, the method comprising:
obtaining a population of cells comprising TCR-NK cells; and
contacting the population of cells with an agent capable of binding to CD3.

In an aspect of the present disclosure, there is provided a method of producing natural killer (NK) cells that comprise one or more exogenous nucleic acids encoding a TCR or portion thereof, the method comprising:
i) culturing NK cells under activating conditions; and
ii) transducing the activated NK cells under conditions to transfer the one or more exogenous nucleic acids to the NK cells, wherein the transduction takes place on the 4th day or later day after the beginning of step i).

In an aspect of the present disclosure, there is provided an NK cell obtained or obtainable by any of the methods disclosed herein. In an aspect of the present disclosure, there is provided a TCR-NK cell obtained or obtainable by any of the methods disclosed herein.

In an aspect of the present disclosure, there is provided a population comprising NK cells for use in a method of treatment, wherein the population has been generated by any of the methods disclosed herein. In an aspect of the present disclosure, there is provided a population comprising TCR-NK cells for use in a method of treatment, wherein the population has been generated by any of the methods disclosed herein.

In an aspect of the present disclosure, there is provided a population comprising TCR-NK cells for use in a method of treatment, wherein, prior to administration, the TCR-NK cells are contacted in vitro with an anti-CD3 agent.

### BRIEF DESCRIPTION OF THE DRAWINGS

FIG. 1 illustrates plots from flow cytometry experiments designed to determine the efficiency of sorting TCR-NK cells with anti-CD3 or anti-TCR.

FIG. 2 illustrates plots from flow cytometry experiments designed to determine the effect of anti-CD3 enrichment on TCR-NK cell surface markers.

FIG. 3 illustrates data from experiments designed to determine the effect of anti-CD3 enrichment on TCR-NK cells transduced to express one of two different TCRs.

FIG. 4 illustrates data from experiments designed to determine the effect of anti-CD3 enrichment on innate and TCR-directed cytotoxicity for TCR-NK cells.

FIG. 5 illustrates data from experiments designed to determine the effect of anti-CD3 enrichment on the degranulation capacity of TCR-NK cells.

FIG. 6 illustrates data from experiments designed to determine the effect of anti-CD3 enrichment on the proliferative capacity of TCR-NK cells.

FIG. 7 illustrates plots from flow cytometry experiments designed to determine a transduction window for a single transduction.

FIG. 8 illustrates data from experiments designed to determine a transduction window for a single transduction.

FIG. 9 illustrates plots from flow cytometry experiments designed to determine a transduction window and to compare a single transduction and a double transduction.

FIG. 10 illustrates flow cytometry plots of TCR-NK cells generated by an exemplary method.

FIG. 11 illustrates the potency of TCR-NK cells generated by an exemplary method.

### DETAILED DESCRIPTION

Provided herein are methods of manufacturing NK cells suitable for therapeutic use. The inventors provide conditions that lead to improved yields of transduced NK cells and populations of NK cells with improved functionalities.

An important property of transduced therapeutic populations of NK cells is the proportion of NK cells within the population that express the transduced genes. The inventors provide herein a step that can be applied post-transduction to enrich the population for transduced cells. However, an enriched population is not preferable if the enrichment has a negative effect on functionality of the population. This is a problem if, for instance, an NK cell population is transduced with a cell surface protein required for therapeutic functionality and the same cell surface protein is used as a marker for enrichment in a manner that blocks said protein. Other complexities can arise if the cell surface protein is capable of sending an intracellular signal when bound by a ligand, and use of such cell surface proteins as markers for enrichment can lead to unpredictable effects on the functionality of the cells.

It is known that CD3 can be associated with the above-mentioned problems. CD3 is normally expressed by T cells and crosslinking of CD3, in the absence of other signals, can lead to negative effects on functionality. Renders and Valerius (Clin Exp Immunol 2003; 133:307-309) report that anti-CD3 antibodies, in the absence of a co-stimulatory signal, can lead to T cell anergy or apoptosis. Schwartz (Science, New Series, Vol. 248, No. 4961 (Jun. 15, 1990), pp. 1349-1356) provides a review of clonal T cell anergy that explains that antigen-receptor signalling in the absence of a co-stimulatory signal is not a neutral event and induces a state of unresponsiveness characterised by an inability to produce IL-2 in subsequent exposures. Chatenoud et al. (Proc. Natl. Acad. Sci. USA Vol. 91, pp. 123-127, January 1994) discuss the use of anti-CD3 monoclonal antibodies as an immunosuppressive treatment, hence revealing the immunosuppressive effects are also seen in vivo. Li and Kurlander (Journal of Translational Medicine 2010, 8:104) also report that contacting T cells with anti-CD3 in the absence of a co-stimulatory signal can result in premature T cell apoptosis or anergy. Li and Kurlander report that the outcomes can be different depending on the nature of the signal and on the history of the cell, hence demonstrating the unpredictability in the field. Thus, the effect of contacting cells with anti-CD3 antibodies on their downstream functionality is unpredictable.

The present inventors have developed a method that enriches for NK cells that have been transduced to express a functional CD3-TCR complex; the method leads to a higher level of enrichment in comparison to alternative approaches. The inventors demonstrate that the method does not negatively affect the functionality of the NK cells and, in some embodiments, have surprisingly found that this method enhances the downstream killing and degranulation performance of the NK cells. This enhancement was present even after a prolonged expansion culture. The method did not negatively affect the expansion profile of the cell population.

Thus, in a first aspect, there is provided a method of enriching TCR-NK cells within a population of cells, the method comprising:
obtaining a population of cells comprising TCR-NK cells; and
enriching for TCR-NK cells using surface CD3 as a marker.

NK cells are known in the art and may be identified by their CD3⁻ CD56⁺ phenotype. TCR-NK cells are CD3⁺ but this is only by virtue of their modification and unmodified NK cells would be CD3⁻. NK cells also have a known morphology in common with other lymphocytes, and this morphology can be identified by the skilled person. For instance, lymphocytes have a large nucleus relative to other types of cell.

As discussed in the background section, TCR-NK cells are those that express a functional CD3-TCR complex. A functional CD3-TCR complex, within the present context, is a CD3-TCR complex that endows the NK cell with the potential for TCR-directed cytotoxicity. The functionality may be measured by techniques known in the art, such as degranulation in the presence of antigen-expressing target cells (but not controls) or killing of antigen-expressing target cells (but not controls). TCR-NK cells are described in WO2016/116601A1 (herein incorporated by reference) and a later publication discussing the TCR-NK approach is WO2018/129199A1 (herein incorporated by reference).

The method of the first aspect is performed in vitro. The method may be performed prior to infusion of the population to a subject. The method may be referred to as being performed ex vivo.

The enrichment may comprise contacting the population with an agent capable of binding to CD3. The agent may bind to any one of CD3γ, CD3δ, or CD3ε. The agent may bind to an epitope that encompasses a region or regions from one or more of CD3γ, CD3δ, and CD3ε. In embodiments of the invention, the agent is capable of binding to CD3, or a particular CD3 chain, as expressed on the TCR-NK cell surface. The agent may be referred to as, or as comprising, an anti-CD3 molecule.

The agent may be or may comprise an antibody or antibody fragment. Thus, the agent may be or may comprise a moiety be based on an antibody scaffold. The antibody or fragment thereof may be capable of binding specifically to CD3 (referred to as “anti-CD3” in the art). The agent may be capable of cross-linking CD3 and may be bivalent or multivalent. The agent may be or may comprise an anti-CD3 antibody, or anti-CD3 fragment thereof, that has or does not have an agonistic effect on T cells.

The agent may comprise a substrate comprising immobilised anti-CD3 molecules. The substrate may be, for instance, a nanomatrix or a bead. In a particular embodiment, the substrate is a bead.

Beads that are suitable for cell sorting and/or cell stimulation are known in the art. In some examples the beads are nanoscale or microscale. For instance, the beads may be 10-100 nm or approximately 50 nm. Alternatively, the beads may be larger, such as approximately 2-5 μM.

Nanomatrices suitable for cell sorting and/or cell stimulation are known in the art. For example, colloidal reagents comprising or consisting of iron oxide crystals embedded into a biocompatible polysaccharide matrix may form nanomatrices that can be coated with proteins, such as antibodies or antibody fragments. The nanomatrix may be, for example, approximately 100 nm in diameter.

The agent may comprise a label that can be physically detected, such as a magnetic moiety. Alternatively, the agent may comprise a moiety that can be bound by an immobilised receptor. Thus, the physically detectable label allows the cells to be separated from a solution or retained in place while a solution is removed. In a particular embodiment, the agent is a magnetic bead.

In an embodiment of the invention, the agent is a magnetic bead comprising immobilised anti-CD3 antibodies or anti-CD3 fragments thereof. Thus, the method of the first aspect may comprise contacting the cells with magnetic beads comprising immobilised anti-CD3 antibodies or anti-CD3 fragments thereof. The step of enriching for cells that are bound by the agent would then comprise contacting the cells with a magnet and separating bead-bound cells from non-bead bound cells. This may involve placing a vessel comprising a cell suspension containing the TCR-NK cells and the beads in the proximity of a magnet. Alternatively, this may comprise applying a cell suspension containing the TCR-NK cells and the beads to a column containing magnets.

Alternative methods of labelling and enriching cells would also be suitable.

The methods may result in a population of cells that is improved for one or more functions. For example, the population may exhibit an increase in innate killing (i.e. conventional NK cell cytotoxicity) in comparison to a control population. Alternatively, or in addition, the population may exhibit an increase in TCR-directed killing in comparison to a control.

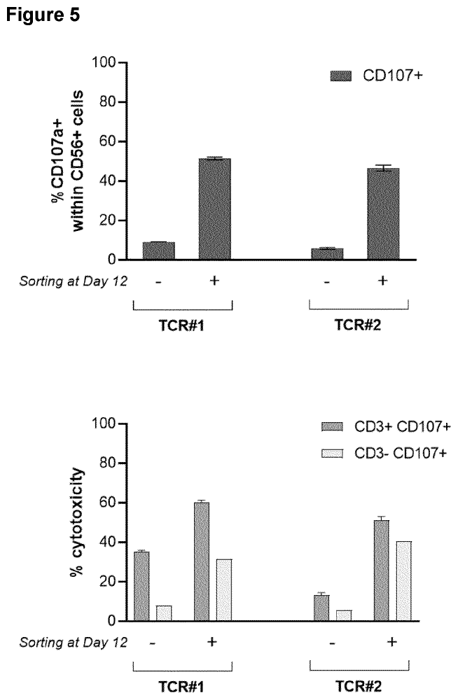

As mentioned above, the inventors have discovered that the contacting of a population of TCR-NK cells with an agent capable of binding to CD3 can be used to improve the functionality of a population of TCR-NK cells. The benefits were present even after a subsequent period of expansion. The inventors demonstrated an improvement in both TCR-directed target cell killing and also in innate cell killing (see, for example, FIG. 5).

Thus, in a second aspect, there is provided a method for enhancing one or more functions of a population of cells comprising TCR-NK cells, the method comprising:
obtaining a population of cells comprising TCR-NK cells; and
contacting the population of cells with an agent capable of binding to CD3.

As used herein, an enhanced population comprising TCR-NK cells is one that has at least one increase in a function. For example, an enhanced population may be one that exhibits an increase in innate killing of target cells (i.e. conventional NK cell cytotoxicity) in comparison to a control population. As another example, an enhanced population may be one that exhibits an increase in TCR-directed killing of target cells in comparison to a control. In particular embodiments, the enhanced population comprises TCR-NK cells that have increased innate killing and TCR-directed killing in comparison to a control that has not been contacted with the agent capable of binding to CD3. Thus, the method of the second aspect may be a method of increasing the innate cytotoxicity and/or the TCR-directed cytotoxicity of a population comprising TCR-NK cells.

The method of the second aspect is performed in vitro. The method may be performed prior to infusion of the population to a subject. The method may be referred to as being performed ex vivo.

In some embodiments, the method further comprises a step of enriching the population for cells that are bound by the agent, as discussed in relation to the first aspect. Thus, the method of the second aspect may be a method of enhancing and enriching a population comprising TCR-NK cells.

NK cells may be identified as discussed in relation to the first aspect. TCR-NK cells are as discussed in relation to the first aspect.

The method of the second aspect involves contacting the cells with an agent capable of binding to CD3. The agent may bind to any one of CD3γ, CD3δ, or CD3ε. The agent may bind to an epitope that encompasses a region or regions from one or more of CD3γ, CD3δ, and CD3ε. In embodiments of the invention, the agent is capable of binding to CD3, or a particular CD3 chain, as expressed on the TCR-NK cell surface. The agent may be referred to as, or as comprising, an anti-CD3 molecule.

The agent may be or may comprise an antibody or antibody fragment. Thus, the agent may be or may comprise a moiety based on an antibody scaffold. The antibody or fragment thereof may be capable of binding specifically to CD3. The agent may or may not be an agonistic agent. An agonistic agent is one capable of binding to CD3 in a manner that transduces a signal that enhances the TCR-NK cell, as discussed herein. The agent may be capable of cross-linking CD3 and may be bivalent or multivalent. The agent may be or may comprise an anti-CD3 antibody, or anti-CD3 fragment thereof, that has or does not have an agonistic effect on T cells.

The agent may be a substrate comprising immobilised anti-CD3 molecules. The substrate may be, for instance, a nanomatrix or a bead. In a particular embodiment, the substrate is a bead. Suitable beads and nanomatrices are discussed in relation to the first aspect.

As discussed in relation to the first aspect, the agent may be suitable for use in an enrichment step. Any of the features disclosed in relation to the enrichment of the first aspect may also be applied to the second aspect.

With regards to either the first aspect or the second aspect of the present disclosure, the TCR-NK cells are modified to express a TCR. Any suitable methods for the generation of a TCR-NK cell may be used and the first and second aspects are not limited to the methods of the sixth aspect of the present disclosure. Nevertheless, the methods of the sixth aspect may be used to generate and expand TCR-NK cell populations in conjunction with the methods of the first and second aspects of the present disclosure.

For the first or the second aspect of the present disclosure, the modification to generate TCR-NK cells may involve the transfer of a construct encoding said TCR to the NK cells. Alternatively, the modification may involve the transfer of a construct encoding said TCR to precursors of said NK cells, which are subsequently converted or differentiated into NK cells. The construct encoding the TCR may be part of a viral vector.

Exemplary viral vectors include retroviral vectors, adenoviral vectors, adeno-associated viral vectors, vaccinia viral vectors, fowlpox viral vectors, and lentiviral vectors. In a particular embodiment, the vector is a lentiviral vector.

NK cells expressing TCRs, with no other modifications, are not functional. The cells must be modified to also express CD3 chains to enable the formation of a functional CD3-TCR complex. In some examples, the NK cells are modified to express a CD3γ chain and a CD3δ chain. In some examples, the NK cells are modified to express a CD3γ chain, a CD3δ chain, and a CD3ε chain. In a particular embodiment, the NK cells are modified to express a CD3γ chain, a CD3δ chain, a CD3ε chain, and a CD3ζ chain.

In some examples, the CD3 chains are part of a fusion protein. For instance, two or more of the CD3 chains may be expressed as part of a single fusion protein. The TCR may also be part of a fusion protein with one, two, three, or all four of the CD3 chains.

The modification of the NK cell may involve the transfer of a construct or constructs encoding the CD3 chains or fusion protein to the NK cells. Alternatively, the modification may involve the transfer of a construct or constructs encoding the CD3 chains or fusion protein to precursors of said NK cells, which are subsequently converted or differentiated into NK cells. The modification may comprise the modification of the NK cell's genome such that the relevant endogenous gene is expressed in the NK cell. The modification may comprise the modification of the NK cell's genome such that the relevant endogenous gene is expressed constitutively or such that the expression may be induced.

The construct or constructs encoding the CD3 chains or fusion protein may be part of a viral vector or viral vectors. Exemplary viral vectors include retroviral vectors, adenoviral vectors, adeno-associated viral vectors, vaccinia viral vectors, fowlpox viral vectors, and lentiviral vectors. In a particular embodiment, the vector is a lentiviral vector.

The construct or constructs encoding the CD3 chains may be transferred separately from the TCR construct. Alternatively, a single construct may encode the TCR and one or more of the CD3 chains. A single construct may encode the TCR and all of the CD3 chains.

The CD3 chains may be transferred via a different vector or vectors from the TCR construct. The CD3 chains may be transferred via the same vector as the TCR construct.

In relation to the first or second aspect of the present disclosure, the TCR-NK cells may be generated from any suitable source of NK cells. The source of NK cells may be a source of primary NK cells. For instance, the cells may be obtained from peripheral blood mononuclear cells (PBMCs) or from cord blood. The NK cells may be sorted or enriched from the source, for instance by positive and/or negative selection. The NK cells may be sorted by depleting all other cells within the source population, including red blood cells, T cells (e.g. using a label for CD3 or TCR), B cells (e.g. using a label for CD19), monocytes (e.g. using a label for CD14), and macrophages (e.g. using a label for CD14). The NK cells may be sorted by selecting for cells expressing CD56. The NK cells may be sorted by depleting CD3⁺ cells and selecting for CD56⁺ cells. The NK cells may be differentiated from precursor cells, such as stem cells. Examples of such stem cells include induced pluripotent stem cells (iPSCs) and haematopoietic stem cells. The stem cells may be CD34⁺ stem cells.

The NK cells may be freshly obtained. The NK cells may be previously obtained and cryopreserved. The source cell population may be previously obtained and cryopreserved, and the NK cells may be isolated after the source cell population is thawed.

To generate TCR-NK cells, NK cells may be transduced after a pre-activation step. For the first and second aspects of the present disclosure, the pre-activation step is not particularly limited and any suitable conditions may be used. For instance, the pre-activation step may comprise culturing the NK cells in the presence of one or more cytokines. As a purely illustrative example, the pre-activation step may comprise culturing the NK cells in the presence of IL-21, IL-12, IL-18, IL-2 and/or IL-15. The pre-activation step may comprise culturing the NK cells in the presence of IL-2 and IL-15. The conditions for pre-activation of NK cells to enable transduction may depend on the transduction method used. Conditions suitable for other methods of transduction are described in the prior art.

The following cytokine concentrations are relevant to the sixth aspect of the present disclosure but, in a non-limiting example, may also be applied to the first and second aspects. The IL-2 may be present at a concentration of at least 1, 10, 100, 200, 250, 500, or 1000 IU/ml. The IL-2 may be present at a concentration of 1-1000 IU/ml, 10-900 IU/ml, 100-800 IU/ml, 250-750 IU/ml, 400-600 IU/ml, 450-550 IU/ml, or approximately 500 IU/ml. The IL-2 may be present at a concentration of approximately 10, 100, 200, 500, or 1000 IU/ml. The IL-2 may be present at a concentration of 220-670 IU/ml, 330-560 IU/ml, 370-520 IU/ml, 410-480 IU/ml, or approximately 440 IU/ml. The IL-2 may be present at a concentration of 370-590 IU/ml or 440-520 IU/ml. The IL-15 may be present at a concentration of at least 0.1, 0.25, 0.5, or 1 ng/ml. The IL-15 may be present at a concentration of at least 0.1, 0.25, 0.5, 1, 2, 3, 4, 5, 6, or 7 ng/ml. The IL-15 may be present at a concentration of 0.1-2 ng/ml, 0.25-1.75 ng/ml, 0.5-1.5 ng/ml, 0.75 to 1.25 ng/ml, or approximately 1 ng/ml. The IL-15 may be present at a concentration of 2-12 ng/ml, 3-11 ng/ml, 4-10 ng/ml, 5-9 ng/ml, 6-8 ng/ml, or approximately 7 ng/ml. The IL-15 may be present at a concentration of 0.5-10, 0.7-9, 0.9-8, or 1-7 ng/ml. In a particular embodiment, the pre-activation is in the presence of approximately 400-600 IU/ml IL-2 and approximately 0.5-1.5 ng/ml IL-15. In a particular embodiment, the pre-activation is in the presence of approximately 370-520 IU/ml IL-2 and approximately 5-9 ng/ml IL-15. In another embodiment, the pre-activation is in the presence of approximately 370-520 or 440-520 IU/ml IL-2 and approximately 0.5-10 or 1-7 ng/ml IL-15. The pre-activation step may be in the presence of feeder cells. In a particular embodiment, the pre-activation step is performed in the presence of IL-2 and/or IL-15 and the absence of feeder cells.

International unit (IU) is a standard measure of cytokine activity. Another standard measure is ng/ml. For the purposes of the present disclosure, IL-2 IU may be converted to ng/ml by assuming a specific activity of 7.4 IU/ng. The cytokine concentrations in the preceding paragraph may be expressed as follows. The IL-2 may be present at a concentration of at least 0.14, 1.4, 14, 27, 34, 68, or 140 ng/ml. The IL-2 may be present at a concentration of 0.14-140 ng/ml, 1.4-120 ng/ml, 14-110 ng/ml, 34-100 ng/ml, 54-81 ng/ml, 61-74 ng/ml, or 50 approximately 68 ng/ml. The IL-2 may be present at a concentration of at least 1.4, 14, 27, 68, or 140 ng/ml. The IL-2 may be present at a concentration of 30-90 ng/ml, 45-75 ng/ml, 50-70 ng/ml, 55-65 ng/ml, or approximately 60 ng/ml. The IL-2 may be present at a concentration of 50-80 ng/ml or 60-70 ng/ml. In a particular embodiment, the pre-activation is in the presence of approximately 54-81 ng/ml IL-2 and approximately 0.5-1.5 ng/ml IL-15. In a particular embodiment, the pre-activation is in the presence of approximately 50-70 ng/ml IL-2 and approximately 5-9 ng/ml IL-15. In a particular embodiment, the pre-activation is in the presence of approximately 50-80 ng/ml or 60-70 ng/ml IL-2 and approximately 0.5-10 or 1-7 ng/ml IL-15. In an embodiment, the pre-activation is in the presence of approximately 60 ng/ml IL-2 and approximately 7 ng/ml IL-15.

The IL-15 concentrations may be expressed as IU. For the purposes of the present disclosure, IL-15 ng may be converted to IU/ml by assuming a specific activity of 12 IU/ng. The cytokine concentrations in the preceding paragraphs may be expressed as follows. The IL-15 may be present at a concentration of at least 1.2, 3, 6, or 12 IU/ml. The IL-15 may be present at a concentration of at least 1.2, 3, 6, 12, 24, 36, 48, 60, 72, or 84 IU/ml. The IL-15 may be present at a concentration of 1.2-24 IU/ml, 3-21 IU/ml, 6-18 IU/ml, 9 to 15 IU/ml, or approximately 12 IU/ml. The IL-15 may be present at a concentration of 24-140 IU/ml, 36-130 IU/ml, 48-120 IU/ml, 60-110 IU/ml, 72-96 IU/ml, or approximately 84 IU/ml. The IL-15 may be present at a concentration of 6-120, 8.4-110, 11-96, or 12-84 IU/ml. In a particular embodiment, the pre-activation is in the presence of approximately 54-81 ng/ml IL-2 and approximately 6-9 IU/ml IL-15. In a particular embodiment, the pre-activation is in the presence of approximately 50-70 ng/ml IL-2 and approximately 60-110 IU/ml IL-15. In a particular embodiment, the pre-activation is in the presence of approximately 50-80 ng/ml or 60-70 ng/ml IL-2 and approximately 6-120 or 12-84 IU/ml IL-15. In an embodiment, the pre-activation is in the presence of approximately 60 ng/ml IL-2 and approximately 84 IU/ml IL-15.

The pre-activation culture may last until the transduction step. The pre-activation culture may last until the first transduction.

For the first and second aspects of the present disclosure, the NK cells may be transduced at any suitable timepoint. The following are non-limiting examples that are particularly relevant to the sixth aspect (discussed herein) but may be applied to the first and second aspects. For example, the NK cells may be transduced on the 4^{th} day or later day after the start of the pre-activation. The NK cells may be transduced on the 4^{th} day, 5^{th} day, 6^{th} day, or 7^{th} day after the start of the pre-activation. The NK cells may be transduced on the 4^{th} day or 5^{th} day after the start of the pre-activation. The NK cells may be transduced on the 5^{th} day after the start of the pre-activation. The transduction may start on a specified day and may be for a prolonged period, e.g. overnight. Day zero is considered to be the day on which the cells are placed into the pre-activation culture; the “1st day after” would be anytime the next day, etc. In some embodiments, if an action takes place on a particular day, then an action that takes place on the following day must take place at least 8 hours, at least 12 hours, or at least 16 hours later. An action that takes place on a following day may take place about 16 to about 32 hours later, or about 24 hours later. The transduction may take place more than 80 hours, more than 88 hours, more than 96 hours, more than 112 hours, more than 120 hours, more than 128 hours, more than 136 hours, more than 128 hours, more than 136 hours, more than 144 hours, more than 160 hours, more than 168 hours, more than 176 hours, or more than 184 hours after the start of the pre-activation. The transduction may take place more than 80 hours, more than 88 hours, more than 96 hours, more than 112 hours, more than 120 hours, more than 128 hours, or more than 136 hours after the start of the pre-activation. The transduction may be 80 to 143 hours, 96 to 136 hours, or 112 to 130 hours after the start of the pre-activation. The transduction may start at a specified time and may be for a prolonged period, e.g. overnight. The transduction may comprise two separate transduction steps. The second transduction may take place on the 6^{th} day or later day after the beginning of the pre-activation. The second may take place on the 6^{th} day or 7^{th} day after the start of the pre-activation. The second transduction may take place on the 7^{th} day after the start of pre-activation. The second transduction may take place more than 128 hours, more than 136 hours, more than 144 hours, more than 160 hours, more than 168 hours, more than 176 hours, or more than 184 hours after the start of the pre-activation. The second transduction may be 128 to 191 hours, 144 to 184 hours, or 160 to 178 hours after the start of the pre-activation. The second transduction may start at a specified time and may be for a prolonged period, e.g. overnight. The first transduction may take place on the 4^{th} day or later day after the beginning of the pre-activation and the second transduction may take place on the 6^{th} day or later day after the beginning of the pre-activation. The first transduction may take place on the 4^{th} day or 5^{th} day after the start of the pre-activation and the second may take place on the 6^{th} day or 7^{th} day after the start of the pre-activation. The first transduction may take place on the 5^{th} day after the start of the pre-activation and the second transduction may take place on the 7^{th} day after the start of pre-activation.

For the first and second aspects of the present disclosure, the technique used to introduce the exogenous nucleic acids is not particularly limited and any suitable technique may be used. For example, viral methods, non-viral methods, transposon techniques, CRISPR-based approaches, techniques involving the introduction of mRNA, and the like. In an embodiment, the transduction steps are performed using a lentiviral vector. The transduction steps may comprise transduction of the same exogenous nucleic acids into cells of the population. The transduction steps may comprise the transfer of one or more exogenous nucleic acids encoding a TCR, a CD3γ chain, a CD3δ chain, a CD3ζ chain, and a CD3ε chain.

The post-transduction conditions for the first and second aspects of the present disclosure may be any suitable. The following are non-limiting examples that are particularly relevant to the sixth aspect (discussed herein) but may be applied to the first and second aspects. For example, after the transduction the population may then be cultured in the presence of feeder cells. Exemplary feeder cells are those expressing membrane-bound IL-21 (mbIL-21) and 4-1BBL. The feeder cells may be K562 cells, 721.221 cells, EBV-LCL cells, NKF cells, or other suitable cells. The feeder cells may be irradiated. The feeder cells may be irradiated K562 cells expressing mbIL-21 and 4-1BBL. The cells may be cultured in the presence of feeder cells for at least 1 day, 2 days, 3 days, or 4 days. The cells may be cultured in the presence of feeder cells for 4 days. The feeder cells, which are optionally K562 cells, may be present at a ratio of 1:1, 1:2, 1:3, or 1:4 (NK:feeder cell). In a particular embodiment, the feeder cells are present in a ratio of 1:2.

The first aspect of the present disclosure comprises a step of enriching for TCR-NK cells using surface CD3 as a marker. The second aspect may also comprise this step. The enrichment step takes place after the NK cells have been transduced to express surface CD3. Thus, the enrichment step takes place after the NY cells have been transduced to express a CD3-TCR complex. The enrichment step may take place after the cells have been cultured in the presence of feeder cells. The enrichment step may take place before an expansion culture.

The second aspect of the present disclosure comprises a step that leads to an enhanced population comprising TCR-NK cells. The first aspect may also comprise this step, which may be the enrichment step. The enhancement takes place after the NK cells have been transduced to express surface CD3. Thus, the enhancement takes place after the NK cells have been transduced to express a CD3-TCR complex. The enhancement may take place after the cells have been cultured in the presence of feeder cells. The enhancement may take place before an expansion culture.

Subsequent to the enrichment and/or enhancement, the cells may be cultured under expansion conditions. For the first and second aspects of the present disclosure, the expansion conditions are not particularly limited and any suitable conditions may be used. Illustrative expansion conditions comprise culturing the cells in the presence of one or more cytokines. For instance, the NK cells may be cultured in the presence of IL-2 and/or IL-15 and may be cultured in the presence of both IL-2 and IL-15. The concentrations of the cytokines may be any suitable, and the following are non-limiting examples. The IL-2 may be present at a concentration of at least 1, 10, 50, 100, 150, 175, 180, or 200 IU/ml. The IL-2 may be present at a concentration of 1-400 IU/ml, 50-350 IU/ml, 100-300 IU/ml, 150-250 IU/ml, 175-225 IU/ml, or approximately 200 IU/ml. The IL-2 may be present at a concentration of 1-400 IU/ml, 50-350 IU/ml, 100-300 IU/ml, 150-210 IU/ml, 170-190 IU/ml, or approximately 180 IU/ml. The IL-2 may be present at a concentration of 37-300 IU/ml, 110-260 IU/ml, 150-220 IU/ml, 160-190 IU/ml, 170-185 IU/ml, or approximately 180 IU/ml. The IL-15 may be present at a concentration of at least 0.1, 0.25, 0.5, 1, or 1.5 ng/ml. The IL-15 may be present at a concentration of 0.1-2 ng/ml, 0.25-1.75 ng/ml, 0.5-1.5 ng/ml, 0.75 to 1.25 ng/ml, or approximately 1 ng/ml. The IL-15 may be present at a concentration of 0.1-3 ng/ml, 1-2 ng/ml, 1.25-1.75 ng/ml, 1.4-1.6 ng/ml, or approximately 1.5 ng/ml. The IL-15 may be present at 0.5-2 ng/ml, 0.75-1.75 ng/ml, or 1-1.5 ng/ml. In a particular embodiment, culturing is in the presence of approximately 150-250 IU/ml IL-2 and approximately 0.5-1.5 ng/ml IL-15. In a particular embodiment, culturing is in the presence of approximately 150-210 IU/ml IL-2 and approximately 1.25-1.75 ng/ml IL-15. In another embodiment, culturing is in the presence of approximately 150-250 IU/ml IL-2 and approximately 0.75-1.75 ng/ml or 1-1.5 ng/ml IL-15. The expansion conditions may also comprise the presence of feeder cells. Feeder cells may be added at the beginning of the expansion and may be re-added to the culture during the expansion. The cells may be cultured at 1×10⁵ to 1×10⁶ viable cells/ml, 1.5×10⁵ to 0.5×10⁵ viable cells/ml, 2×10⁵ to 3×10⁵ viable cells/ml, or approximately 2.5×10⁵ viable cells/ml.

As discussed herein, for the purposes of the present disclosure, IL-2 IU may be converted to ng/ml by assuming a specific activity of 7.4 IU/ng. The cytokine concentrations in the preceding paragraph may be expressed as follows. The IL-2 may be present at a concentration of at least 0.14, 1.4, 6.8, 14, 20, 24, or 27 ng/ml. The IL-2 may be present at a concentration of 1-54 ng/ml, 6.8-47 ng/ml, 14-41 ng/ml, 20-34 ng/ml, 24-30 ng/ml, or approximately 27 ng/ml. The IL-2 may be present at a concentration of 0.14-54 ng/ml, 6.8-47 ng/ml, 14-41 ng/ml, 20-28 ng/ml, 23-26 ng/ml, or approximately 24 ng/ml. The IL-2 may be present at a concentration of 5-40 ng/ml, 15-35 ng/ml, 20-30 ng/ml, 22-26 ng/ml, 23-25 ng/ml, or approximately 24 ng/ml. In a particular embodiment, culturing is in the presence of approximately 20-34 ng/ml IL-2 and approximately 0.5-1.5 ng/ml IL-15. In a particular embodiment, culturing is in the presence of approximately 20-30 ng/ml IL-2 and approximately 1.25-1.75 ng/ml IL-15. In another embodiment, culturing is in the presence of approximately 20-35 ng/ml IL-2 and approximately 0.75-1.75 ng/ml or 1-1.5 ng/ml IL-15. In an embodiment, culturing is in the presence of approximately 24 ng/ml IL-2 and approximately 1.5 ng/ml IL-15.

As discussed herein, for the purposes of the present disclosure, IL-15 ng may be converted to IU/ml by assuming a specific activity of 12 IU/ng. The cytokine concentrations in the preceding paragraphs may be expressed as follows. The IL-15 may be present at a concentration of at least 1.2, 3, 6, 12, or 18 IU/ml. The IL-15 may be present at a concentration of 1.2-24 IU/ml, 3-21 IU/ml, 6-18 IU/ml, 9-15 IU/ml, or approximately 12 IU/ml. The IL-15 may be present at a concentration of 1.2-36 IU/ml, 12-24 IU/ml, 15-21 IU/ml, 17-19 IU/ml, or approximately 18 IU/ml. The IL-15 may be present at 6-24 IU/ml, 9-21 IU/ml, or 12-18 IU/ml. In a particular embodiment, culturing is in the presence of approximately 150-250 IU/ml IL-2 and approximately 6-18 IU/ml IL-15. In a particular embodiment, culturing is in the presence of approximately 150-210 IU/ml IL-2 and approximately 15-21 IU/ml IL-15. In another embodiment, culturing is in the presence of approximately 150-250 IU/ml IL-2 and approximately 9-21 IU/ml or 12-18 IU/ml IL-15.

The cells may be expanded in any suitable culture vessel. For instance, the cells may be expanded in a bioreactor. Examples of bioreactors include a rocking bed bioreactor, a stirred tank bioreactor, a rotating wall bioreactor, a perfusion bioreactor, an isolation/expansion automated system, an automated or semi-automated bioreaction, a disposable bag bioreaction, and a gas permeable rapid expansion system. In particular, the bioreaction may be a gas permeable rapid expansion system, for instance the cells may be expanded using a G-Rex™ system. The cells may be expanded for, or for at least, 1, 2, 3, 4, 5, 6, 7, or 8 days. The cells may be expanded for, or for at least, 5, 6, 7, 8, 9, 10, 12, 14, or 16 days. In a particular embodiment, the cells are expanded for 8 days or 16 days. The cell culture media may be refreshed during the culture. As an example, the media may be refreshed every 3 days during the culturing under expansion conditions. For instance, the media may be refreshed 3 days and 6 days into the culturing under expansion conditions. The cells may be expanded for, or for at least, 10, 15, 20, 25, 30, 40, 50, 60, 70, 80, 90, or 100 days. Cytokines and/or feeder cells may be periodically refreshed during the culture. For instance, the cells may be expanded for 70 days and the culture may be pulsed five times with feeder cells. In an embodiment, the cells are expanded for 16 days and the cytokines are replenished 3 days into the expansion, the cytokines are replenished and feeder cells are added 6 days into the expansion, the medium is exchanged (e.g. 75% of the medium is exchanged) 9 days into the expansion, and/or the cytokines are replenished 12 days into the expansion.

The methods of the first or second aspect may further comprise the cryopreservation of the cell population. The methods may further comprise the cryopreservation of the TCR-NK cells. Such cells may subsequently be thawed, resuspended, and formulated as a pharmaceutical composition. The cells may be washed prior to formulation to ensure purity of the pharmaceutical composition. The cryopreservation may take place after the enhanced and/or enriched cells have been expanded.

The methods of the first or second aspect may comprise formulating the TCR-NK cells into a pharmaceutical composition without a prior cryopreservation step. The formulation may take place after the enhanced and/or enriched cells have been expanded.

The pharmaceutical composition may be a cell suspension suitable for administration, for instance via intra-venous injection, into a subject.

In a particular embodiment, the method of the first or second aspect comprises:
activating a population of NK cells in the presence of one or more cytokines,
transducing the population of NK cells to generate TCR-NK cells,
culturing the cells in the presence of feeder cells,
contacting the population of cells with an agent capable of binding to CD3,
enriching the population for cells that are bound by the agent,
expanding the enriched cells.

The TCR-NK cells may, in some examples, be obtained by any method of the sixth aspect of the present disclosure. Any of the features of the sixth aspect, where relevant to TCR-NK cells, may be applied to the first aspect or the second aspect.

In a third aspect, there is provided a population of or comprising TCR-NK cells that is obtained or obtainable by any of the methods of the first aspect or the second aspect of the present disclosure.

In a fourth aspect, there is provided a population of or comprising TCR-NK cells of the third aspect for use in a method of treatment.

In an embodiment, there is provided a method of treating a subject in need thereof, comprising administering a therapeutically effective amount of a population according to the third or the fourth aspect to the subject.

The inventors have discovered that methods comprising contacting TCR-NK cells with an anti-CD3 agent can be used to increase the therapeutic potential of TCR-NK cells. Thus, in a fifth aspect, there is provided TCR-NK cells for use in a method of treatment, wherein, prior to administration, the TCR-NK cells are contacted in vitro with an anti-CD3 agent.

In an embodiment, there is provided a method of treating a subject in need thereof, comprising administering a therapeutically effective amount of a population comprising TCR-NK cells to the subject, and wherein, prior to administration, the TCR-NK cells were contacted in vitro with an anti-CD3 agent.

The methods of contacting the TCR-NK cells with an anti-CD3 agent may be any as discussed in relation to the first or second aspects. For instance, the anti-CD3 agent may be a bead, such as a magnetic bead, comprising immobilised anti-CD3 antibodies or anti-CD3 fragments thereof.

The TCR-NK cells for use of the fifth aspect may be obtained or obtainable by any of the methods of the first, second, or sixth aspect. For instance, a population comprising NK cells may be activated in the presence of cytokines, transduced to generate TCR-NK cells, cultured in the presence of feeder cells, enriched and/or enhanced using the anti-CD3 agent, and expanded.

The population of expanded TCR-NK cells for use of the fourth or fifth aspects may be administered to a subject in need thereof. The subject may be a mammalian subject. The subject may be a mouse, rat, rabbit, dog, cat, horse, or pig. Preferably, the subject is a human.

The methods of treatment of the fourth or fifth aspects may be for the treatment of cancer. The cancers that may be treated comprise cells expressing an antigen that may be targeted by a TCR. Illustrative cancers include lung cancer, head and neck cancer, liver cancer, skin cancer, renal cell cancer, brain cancer, gastric cancer, colorectal cancer, hepatocellular cancer, pancreatic cancer, prostate cancer, leukemia, breast cancer, Merkel cell carcinoma, urinary bladder cancer, uterine cancer, gallbladder and bile duct cancer, osteosarcoma, liposarcoma, a neuroblastoma, a myeloma, a synovial sarcoma, an esophageal cancer, an esophageal squamous cell carcinoma, an ovarian cancer, an ovarian epithelial cancer, an astrocytic tumor, a glioblastoma multiforme, an anaplastic astrocytoma, a fallopian tube cancer, primary peritoneal cavity cancer, advanced solid tumors, soft tissue sarcoma, a sarcoma, a myelodysplastic syndrome, an acute myeloid leukemia, a Hodgkin lymphoma, a non-Hodgkin lymphoma, a Hodgkin disease, a multiple myeloma, a metastatic solid tumor, HPV+ cancer, a stomach cancer, a rhabdomyosarcoma, a myxoid round cell liposarcoma, gastrointestinal stromal cancer (GIST), bronchial cancer, central nervous system cancer, peripheral nervous system cancer, endometrial cancer, cancer of the oral cavity or pharynx, kidney cancer, testicular cancer, biliary tract cancer, small bowel cancer, appendix cancer, salivary gland cancer, thyroid cancer, adrenal gland cancer, chondrosarcoma, or cancer of hematological tissues.

The methods of treatment for the fourth or fifth aspects may be for the treatment of infection by a pathogen. The pathogen infections that may be treated are those that are associated with antigens that may be targeted by TCRs. Illustrative pathogens include viruses, such as epstein-Barr virus (EBV), SARS-CoV-2, hepatitis B virus (HBV), human papillomavirus (HPV), and human immunodeficiency virus (HIV).

The methods of treatment disclosed herein include prophylactic or preventative treatment (e.g. treatment before the onset of a condition in an individual to reduce the risk of the condition occurring in the individual; delay its onset; or reduce its severity after onset).

The terms “prophylactic”, “preventative”, or “preventing” and the like refer to reducing the probability of developing a disorder or condition in a subject, who does not have, but is at risk of or susceptible to developing a disorder or condition. Prevention and the like do not mean preventing a subject from ever getting the specific disease or disorder. Prevention may require the administration of multiple doses. Prevention can include the prevention of a recurrence of a disease in a subject for whom all disease symptoms were eliminated, or prevention of recurrence in a relapsing-remitting disease.

Administration is normally in a “therapeutically effective amount”, this being sufficient to show benefit to a patient. Such benefit may be at least amelioration of at least one symptom. The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners. Prescription of treatment, e.g. decisions on dosage etc., is within the responsibility of medical professionals and may depend on the severity of the symptoms and/or progression of a disease being treated. A therapeutically effective amount or suitable dose of a medicament may be determined by comparing in vitro activity and in vivo activity in an animal model. Methods for extrapolation of effective dosages in mice and other test animals to humans are known. The precise dose will depend upon a number of factors, including whether the medicament is for prevention or for treatment, the size and location of the area to be treated and the precise nature of the medicament.

Administration may be via any suitable method, such as intra-venous administration, e.g. by injection. Administration may be of a single dose or may be repeated periodically.

The methods of the present disclosure may be monotherapies or combination therapies with other agents. The administration of combination therapies may be either simultaneously or sequentially.

An important property of transduced therapeutic populations of NK cells is the proportion of NY cells within the population that express the transduced genes. The level of transduction is a contributing factor to this property. The inventors provide herein improved conditions for the transduction of NK cells with nucleic acid sequences encoding a TCR or a portion thereof. In particular, the inventors have identified a transduction “window” for optimal transduction of NK cells.

Thus, in a sixth aspect, there is provided a method of producing natural killer (NK) cells that comprise one or more exogenous nucleic acids encoding a TCR or portion thereof, the method comprising:
i) culturing NK cells under activating conditions; and
ii) transducing the activated NY cells under conditions to transfer the one or more exogenous nucleic acids to the NK cells, wherein the transduction takes place on the 4th day or later day after the beginning of step i).

NK cells may be identified as discussed in relation to the first aspect. The NK cells that are cultured in step i) may be from any suitable source of NK cells. The source may be a source of primary NK cells. For instance, the cells may be obtained from peripheral blood mononuclear cells (PBMCs) or from cord blood. The NK cells may be sorted or enriched from the source, for instance by positive and/or negative selection. The NK cells may be sorted by depleting all other cells within the source population, including red blood cells, T cells (e.g. using a label for CD3 or TCR), B cells (e.g. using a label for CD19), monocytes (e.g. using a label for CD14), and macrophages (e.g. using a label for CD14). The NK cells may be sorted by selecting for cells expressing CD56. The NK cells may be sorted by depleting CD3⁺ cells and selecting for CD56⁺ cells. The NK cells may be differentiated from precursor cells, such as stem cells. Examples of such stem cells include induced pluripotent stem cells (iPSCs) and haematopoietic stem cells. The stem cells may be CD34⁺ stem cells. The NK cells may be freshly obtained. The NK cells may be previously obtained and cryopreserved. The source cell population may be previously obtained and cryopreserved, and the NK cells may be isolated after the source cell population is thawed.

Step i) may be considered a “pre-activation” step. The pre-activation may comprise culturing the NK cells in the presence of one or more cytokines. For instance, the NK cells may be cultured in the presence of IL-2 and/or IL-15. In a particular embodiment, the NK cells are cultured in the presence of both IL-2 and IL-15.

The IL-2 may be present at a concentration of at least 1, 10, 100, 250, 500, or 1000 IU/ml. The IL-2 may be present at a concentration of 1-1000 IU/ml, 10-900 IU/ml, 100-800 IU/ml, 250-750 IU/ml, 400-600 IU/ml, 450-550 IU/ml, or approximately 500 IU/ml. The IL-2 may be present at a concentration of approximately 10, 100, 200, 500, or 1000 IU/ml. The IL-2 may be present at a concentration of 220-670 IU/ml, 330-560 IU/ml, 370-520 IU/ml, 410-480 IU/ml, or approximately 440 IU/ml. The IL-2 may be present at a concentration of 370-590 IU/ml or 440-520 IU/ml.

As discussed herein, IL-2 amounts may be expressed in ng/ml. The IL-2 may be present at a concentration of at least 0.14, 1.4, 14, 34, 68, or 140 ng/ml. The IL-2 may be present at a concentration of 0.14-140 ng/ml, 1.4-120 ng/ml, 14-110 ng/ml, 34-100 ng/ml, 54-81 ng/ml, 61-74 ng/ml, or approximately 68 ng/ml. The IL-2 may be present at a concentration of at least 1.4, 14, 27, 68, or 140 ng/ml. The IL-2 may be present at a concentration of 30-90 ng/ml, 45-75 ng/ml, 50-70 ng/ml, 55-65 ng/ml, or approximately 60 ng/ml.

The IL-2 may be present at a concentration of 50-80 ng/ml or 60-70 ng/ml.

The IL-15 may be present at a concentration of at least 0.1, 0.25, 0.5, or 1 ng/ml. The IL-15 may be present at a concentration of 0.1-2 ng/ml, 0.25-1.75 ng/ml, 0.5-1.5 ng/ml, 0.75 to 1.25 ng/ml, or approximately 1 ng/ml. The IL-15 may be present at a concentration of at least 0.1, 0.25, 0.5, 1, 2, 3, 4, 5, 6, or 7 ng/ml. The IL-15 may be present at a concentration of 2-12 ng/ml, 3-11 ng/ml, 4-10 ng/ml, 5-9 ng/ml, 6-8 ng/ml, or approximately 7 ng/ml.

The IL-15 may be present at a concentration of 0.5-10, 0.7-9, 0.9-8, or 1-7 ng/ml.

In a particular embodiment, the pre-activation is in the presence of approximately 400-600 IU/ml IL-2 and approximately 0.5-1.5 ng/ml IL-15. In another embodiment, the pre-activation is in the presence of approximately 370-520 IU/ml IL-2 and approximately 5-9 ng/ml IL-15. In another embodiment, the pre-activation is in the presence of approximately 370-520 or 440-520 IU/ml IL-2 and approximately 0.5-10 or 1-7 ng/ml IL-15. In a particular embodiment, the pre-activation is in the presence of approximately 54-81 ng/ml IL-2 and approximately 0.5-1.5 ng/ml IL-15. In a particular embodiment, the pre-activation is in the presence of approximately 50-70 ng/ml IL-2 and approximately 5-9 ng/ml IL-15.

In a particular embodiment, the pre-activation is in the presence of approximately 50-80 ng/ml or 60-70 ng/ml IL-2 and approximately 0.5-10 ng/ml or 1-7 ng/ml IL-15. In an embodiment, the pre-activation is in the presence of approximately 60 ng/ml IL-2 and approximately 7 ng/ml IL-15.

As discussed herein, for the purposes of the present disclosure, IL-15 ng may be converted to IU/ml. The IL-15 concentrations in the preceding paragraphs may be expressed as follows. The IL-15 may be present at a concentration of at least 1.2, 3, 6, or 12 IU/ml. The IL-15 may be present at a concentration of 1.2-24 IU/ml, 3-21 IU/ml, 6-18 IU/ml, 9-15 IU/ml, or approximately 12 IU/ml. The IL-15 may be present at a concentration of at least 1.2, 3, 6, 12, 24, 36, 48, 60, 72, or 84 IU/ml. The IL-15 may be present at a concentration of 24-144 IU/ml, 36-132 IU/ml, 48-120 IU/ml, 60-110 IU/ml, 72-96 IU/ml, or approximately 84 IU/ml. The IL-15 may be present at a concentration of 6-120, 8.4-110, 11-96, or 12-84 IU/ml. In a particular embodiment, the pre-activation is in the presence of approximately 400-600 IU/ml IL-2 and approximately 6-18 IU/ml IL-15. In another embodiment, the pre-activation is in the presence of approximately 370-520 IU/ml IL-2 and approximately 60-110 IU/ml IL-15.

In another embodiment, the pre-activation is in the presence of approximately 370-520 or 440-520 IU/ml IL-2 and approximately 6-120 or 12-84 IU/ml IL-15. In a particular embodiment, the pre-activation is in the presence of approximately 54-81 ng/ml IL-2 and approximately 6-18 IU/ml IL-15. In a particular embodiment, the pre-activation is in the presence of approximately 50-70 ng/ml IL-2 and approximately 60-110 IU/ml IL-15. In a particular embodiment, the pre-activation is in the presence of approximately 50-80 ng/ml or 60-70 ng/ml IL-2 and approximately 6-120 IU/ml or 12-84 IU/ml IL-15. In an embodiment, the pre-activation is in the presence of approximately 60 ng/ml IL-2 and approximately 84 IU/ml IL-15.

During the pre-activation, the cells may be cultured at 1×10⁵ to 5×10⁶ viable cells/ml, 2.5×10⁵ to 2.5×10⁶ viable cells/ml, 5×10⁵ to 1.5×10⁶ viable cells/ml, 7.5×10⁵ to 1.25×10⁶ viable cells/ml, or approximately 1×10⁶ viable cells/ml. The cells may be cultured at 0.5 to 1×10⁶ cells/cm².

In a preferred embodiment, step i) is in the absence of feeder cells. Step i) may last until the transduction step. Step i) may last until the first transduction.

Step ii) may comprise the transduction of the cells with one or more nucleic acids encoding a T cell receptor (TCR) and/or one or more CD3 chains. As discussed in relation to the first and second aspects, in some examples the NK cells are modified to express a CD3γ chain and a CD3δ chain; a CD3γ chain, a CD3δ chain, and a CD3ε chain; or a CD3γ chain, a CD3δ chain, a CD3ε chain, and a CD3ζ chain. In some examples, the CD3 chains are part of a fusion protein, e.g., two or more of the CD3 chains may be expressed as part of a single fusion protein. The TCR may also be part of a fusion protein with one, two, three, or all four of the CD3 chains. Step ii) may comprise the transduction of the cells with one or more nucleic acids encoding a TCR, a CD3γ chain, a CD3δ chain, a CD3ζ chain, and a CD3ε chain. The transduction may be to generate TCR-NK cells as discussed herein. The discussion of TCR-NK cells in relation to the first or second aspects is also relevant to the sixth aspect.

Alternatively, step ii) may comprise the transduction of the cells with one or more nucleic acids encoding a CAR, wherein the CAR comprises TCR or an antigen-binding fragment thereof (referred to herein as a “TCR-CAR”). One exemplary approach is disclosed in WO2019/069125A1 (herein incorporated by reference).

Step ii) may be a single transduction or may comprise a first transduction. The NK cells may be transduced on the 4^{th} day or later day after the start of the pre-activation. The NK cells may be transduced on the 4^{th} day, 5^{th} day, 6^{th} day, or 7^{th} day after the start of the pre-activation. The NK cells may be transduced on the 4^{th} day or 5^{th} day after the start of the pre-activation. The NK cells may be transduced on the 5^{th} day after the start of the pre-activation. Day zero is considered to be the day on which the cells are placed into the pre-activation culture; the “1 st day after” would be anytime the next day, etc. In some embodiments, if an action takes place on a particular day then an action that takes place on the following day must take place at least 8 hours, at least 12 hours, or at least 16 hours later. An action that takes place on a following day may take place about 16 to about 32 hours later, or about 24 hours later. The transduction may start on a specified day and may be for a prolonged period, e.g. overnight.

The transduction may take place more than 80 hours, more than 88 hours, more than 96 hours, more than 112 hours, more than 120 hours, more than 128 hours, more than 136 hours, more than 128 hours, more than 136 hours, more than 144 hours, more than 160 hours, more than 168 hours, more than 176 hours, or more than 184 hours after the start of the pre-activation. The transduction may take place more than 80 hours, more than 88 hours, more than 96 hours, more than 112 hours, more than 120 hours, more than 128 hours, or more than 136 hours after the start of the pre-activation. The first transduction may be 80 to 143 hours, 96 to 136 hours, or 112 to 130 hours after the start of the pre-activation. The transduction may start at a specified time and may be for a prolonged period, e.g. overnight.

The single transduction or first transduction may comprise the use of two viral vectors; one for the transfer of a nucleic acid encoding a TCR and the other for the transfer of a nucleic acid encoding CD3 chains, for instance all four CD3 chains.

Step ii) may be a double transduction and so may comprise two separate transduction steps. The second transduction may take place on the 6^{th} day or later day after the beginning of the pre-activation. The second may take place on the 6^{th} or 7^{th} day after the start of the pre-activation. The second transduction may take place on the 7^{th} day after the start of pre-activation. The second transduction may take place more than 128 hours, more than 136 hours, more than 144 hours, more than 160 hours, more than 168 hours, more than 176 hours, or more than 184 hours after the start of the pre-activation. The second transduction may be 128 to 191 hours, 144 to 184 hours, or 160 to 178 hours after the start of the pre-activation. The second transduction may start on specified day or at a specified time and may be for a prolonged period, e.g. overnight.

The second transduction may comprise the use of two viral vectors: one for the transfer of a nucleic acid encoding a TCR and the other for the transfer of a nucleic acid encoding CD3 chains, for instance all four CD3 chains.

The first transduction may take place on the 4^{th} day or later day after the beginning of the pre-activation and the second transduction may take place on the 6^{th} day or later day after the beginning of the pre-activation. The first transduction may take place on the 4^{th} or 5^{th} day after the start of the pre-activation and the second may take place on the 6^{th} day or 7^{th} day after the start of the pre-activation. The first transduction may take place on the 5^{th} day after the start of the pre-activation and the second transduction may take place on the 7^{th} day after the start of pre-activation.

The first and second transduction steps may comprise transduction of the same exogenous nucleic acids into cells of the population.

In a preferred embodiment, the transduction steps are performed using one or more lentiviral vectors. The lentiviral vector may be a VSV-g pseudotyped lentiviral vector. In an embodiment, step ii) comprises two transduction steps both of which comprise the lentiviral transfer of one or more exogenous nucleic acids encoding a TCR and one or more CD3 chains. In an embodiment, step ii) comprises two transduction steps both of which comprise the lentiviral transfer of one or more exogenous nucleic acids encoding a TCR, a CD3γ chain, a CD3δ chain, a CD3ζ chain, and a CD3ε chain. Each transduction step may comprise the use of two lentiviral vectors: one for the transfer of a nucleic acid encoding a TCR and the other for the transfer of a nucleic acid encoding the CD3 chains. In a particularly preferred embodiment, step ii) comprises two lentiviral transduction steps both of which comprise the use of two lentiviral vectors: one for the transfer of a nucleic acid encoding a TCR and the other for the transfer of a nucleic acid encoding a CD3γ chain, a CD3δ chain, a CD3ζ chain, and a CD3ε chain, wherein the first transduction take place on the 5^{th} day after the start of the pre-activation and the second transduction takes place on the 7^{th} day after the start of pre-activation.

During transduction, the cells may be at 1×10⁵ to 5×10⁶ viable cells/ml, 2.5×10⁵ to 2.5×10⁶ viable cells/ml, 5×10⁵ to 1.5×10⁶ viable cells/ml, 7.5×10⁵ to 1.25×10⁶ viable cells/ml, or approximately 1×10⁶ viable cells/ml. The lentiviral vectors may be at a multiplicity of infection (MOI) of 1 to 35, 10 to 30, 15 to 25, or 20.

The transduction may take place in the presence of a transduction enhancer, such as protamine sulphate, Lentiboost, Retronectin, cyclosporin A, cyclosporin H, dasatinib, 16,16-Dimethyl Prostaglandin E2, prostaglandin E₂, rapamycin, rosuvastatin calcium, staurosporine, or BX795. In particular, the transduction may take place in the presence of BX795. The BX795 may be present at 1-10 μM, 1.5-7.5 μM, 2-6 μM, or 2.5-5 μM. The BX795 may be present at 1 μM, 2 μM, 2.5 μM, 3 μM, 4 μM, 5 μM, or 6 μM, or any values in-between. The BX795 may be present at approximately 2.5 μM or 5 μM. The transduction may take place in the presence of protamine sulfate, for instance 10 μg/ml of protamine sulfate. The transduction may be a spinoculation optionally followed by a static incubation. The temperature during the transduction may be 32° C.

In-between and optionally for a period after the transduction, the NK cells may be cultured in the presence of one or more cytokines. For instance, the NK cells may be cultured in the presence of IL-2 and/or IL-15. In a particular embodiment, the NK cells are cultured in the presence of both IL-2 and IL-15. The IL-2 may be present at a concentration of at least 1, 10, 50, 100, 150, 175, 180, or 200 IU/ml. The IL-2 may be present at a concentration of 1-400 IU/ml, 50-350 IU/ml, 100-300 IU/ml, 150-250 IU/ml, 175-225 IU/ml, or approximately 200 IU/ml. The IL-2 may be present at a concentration of 1-400 IU/ml, 50-350 IU/ml, 100-300 IU/ml, 150-210 IU/ml, 170-190 IU/ml, or approximately 180 IU/ml. The IL-2 may be present at a concentration of 37-300 IU/ml, 110-260 IU/ml, 150-220 IU/ml, 160-190 IU/ml, 170-185 IU/ml, or approximately 180 IU/ml. As discussed herein, IL-2 amounts may be expressed in ng/ml. The IL-2 may be present at a concentration of at least 0.14, 1.4, 6.8, 14, 20, 24, or 27 ng/ml. The IL-2 may be present at a concentration of 1-54 ng/ml, 6.8-47 ng/ml, 14-41 ng/ml, 20-34 ng/ml, 24-30 ng/ml, or approximately 27 ng/ml. The IL-2 may be present at a concentration of 0.14-54 ng/ml, 6.8-47 ng/ml, 14-41 ng/ml, 20-28 ng/ml, 23-26 ng/ml, or approximately 24 ng/ml. The IL-2 may be present at a concentration of 5-40 ng/ml, 15-35 ng/ml, 20-30 ng/ml, 22-26 ng/ml, 23-25 ng/ml, or approximately 24 ng/ml. The IL-15 may be present at a concentration of at least 0.1, 0.25, 0.5, 1, or 1.5 ng/ml. The IL-15 may be present at a concentration of 0.1-2 ng/ml, 0.25-1.75 ng/ml, 0.5-1.5 ng/ml, 0.75 to 1.25 ng/ml, or approximately 1 ng/ml. The IL-15 may be present at a concentration of 0.1-3 ng/ml, 1-2 ng/ml, 1.25-1.75 ng/ml, 1.4-1.6 ng/ml, or approximately 1.5 ng/ml. The IL-15 may be present at 0.5-2 ng/ml, 0.75-1.75 ng/ml, or 1-1.5 ng/ml. In a particular embodiment, culturing is in the presence of approximately 150-250 IU/ml IL-2 and approximately 0.5-1.5 ng/ml IL-15. In a particular embodiment, culturing is in the presence of approximately 150-210 IU/ml IL-2 and approximately 1.25-1.75 ng/ml IL-15. In another embodiment, culturing is in the presence of approximately 150-250 IU/ml IL-2 and approximately 0.75-1.75 ng/ml or 1-1.5 ng/ml IL-15. In a particular embodiment, culturing is in the presence of approximately 20-34 ng/ml IL-2 and approximately 0.5-1.5 ng/ml IL-15. In a particular embodiment, culturing is in the presence of approximately 20-30 ng/ml IL-2 and approximately 1.25-1.75 ng/ml IL-15. In another embodiment, culturing is in the presence of approximately 20-35 ng/ml IL-2 and approximately 0.75-1.75 ng/ml or 1-1.5 ng/ml IL-15. In an embodiment, culturing is in the presence of approximately 24 ng/ml IL-2 and approximately 1.5 ng/ml IL-15.

As discussed herein, for the purposes of the present disclosure, IL-15 ng may be converted to IU/ml. The IL-15 concentrations in the preceding paragraphs may be expressed as follows. The IL-15 may be present at a concentration of at least 1.2, 3, 6, 12, or 18 IU/ml. The IL-15 may be present at a concentration of 1.2-24 IU/ml, 3-21 IU/ml, 6-18 IU/ml, 9-15 IU/ml, or approximately 12 IU/ml. The IL-15 may be present at a concentration of 1.2-36 IU/ml, 12-24 IU/ml, 15-21 IU/ml, 17-19 IU/ml, or approximately 18 IU/ml. The IL-15 may be present at 6-24 IU/ml, 9-21 IU/ml, or 12-18 IU/ml. In a particular embodiment, culturing is in the presence of approximately 150-250 IU/ml IL-2 and approximately 6-18 IU/ml IL-15. In a particular embodiment, culturing is in the presence of approximately 150-210 IU/ml IL-2 and approximately 15-21 IU/ml IL-15. In another embodiment, culturing is in the presence of approximately 150-250 IU/ml IL-2 and approximately 9-21 IU/ml or 12-18 IU/ml IL-15. In a particular embodiment, culturing is in the presence of approximately 20-34 ng/ml IL-2 and approximately 6-18 IU/ml IL-15. In a particular embodiment, culturing is in the presence of approximately 20-30 ng/ml IL-2 and approximately 15-21 IU/ml IL-15. In another embodiment, culturing is in the presence of approximately 20-35 ng/ml IL-2 and approximately 9-21 IU/ml or 12-18 IU/ml IL-15. In an embodiment, culturing is in the presence of approximately 24 ng/ml IL-2 and approximately 18 IU/ml IL-15.

The cells may be cultured at 1×10⁵ to 5×10⁶ viable cells/ml, 2.5×10⁵ to 2.5×10⁶ viable cells/ml, 5×10⁵ to 1.5×10⁶ viable cells/ml, 7.5×10⁵ to 1.25×10⁶ viable cells/ml, or approximately 1×10⁶ viable cells/ml. The cells may be cultured at 0.125-0.25×10⁶ cell/cm².

After the transduction, the enriched population may be cultured in the presence of feeder cells. Exemplary feeder cells are those expressing mbIL-21 and 4-1BBL. The feeder cells may be K562 cells, 721.221 cells, EBV-LCL cells, NKF cells, or other suitable cells. The feeder cells may be irradiated. The feeder cells may be irradiated K562 cells that express 4-1BBL and mbIL-21. The cells may be cultured in the presence of feeder cells for at least 1 day, 2 days, 3 days, or 4 days. The cells may be cultured in the presence of feeder cells for 4 days. The feeder cells, which are optionally K562 cells, may be present at a ratio of 1:1, 1:2, 1:3, or 1:4 (NK:feeder cell). In a particular embodiment, the feeder cells are irradiated K562 cells that express 4-1BBL and mbIL-21 and are present in a ratio of 1:2. The cells may be cultured at 1×10⁵ to 1×10⁶ viable cells/ml, 1.5×10⁵ to 0.5×10⁵ viable cells/ml, 2×10⁵ to 3×10⁵ viable cells/ml, or approximately 2.5×10⁵ viable cells/ml. The cells may be cultured at 0.125-0.25×10⁶ cell/cm².

The cells may be incubated in any suitable culture vessel. For instance, the cells may be expanded in a bioreactor. Examples of bioreactors include a rocking bed bioreactor, a stirred tank bioreactor, a rotating wall bioreactor, a perfusion bioreactor, an isolation/expansion automated system, an automated or semi-automated bioreaction, a disposable bag bioreaction, and a gas permeable rapid expansion system. In particular, the bioreaction may be a gas permeable rapid expansion system, for instance the cells may be incubated in a G-Rex™ system. The cells may be incubated in a gas permeable rapid expansion system for 4 days in the presence of irradiated K562 cells that express 4-1BBL and mbIL-21 at an NK:feeder cell ratio of 1:2.

The method of the sixth aspect may comprise an enrichment step. The enrichment step may comprise the contacting of the cells after transduction, and optionally after culture with feeder cells, with an agent capable of binding to anti-CD3 or anti-TCR. The enrichment step may be as discussed in relation to the first or second aspects of the present disclosure. The anti-TCR agent may be or may comprise an antibody or an antibody fragment. Thus, the anti-TCR agent may be or may comprise a moiety based on an antibody scaffold. The agent may be a substrate to which anti-TCR molecules are immobilised. The substrate may be, for instance, a nanomatrix or a bead. In a particular embodiment, the substrate is a bead.

The population of cells may be enhanced, as discussed in relation to the second aspect. All features of this step and downstream steps discussed in relation to the second aspect are also applicable to the sixth aspect. The enrichment step may take place after transduction, and optionally after culture with feeder cells.

The agent used during the enrichment and/or enhancement steps may comprise a label that can be physically detected, such as a magnetic moiety. Alternatively, the agent may comprise a moiety that can be bound by an immobilised receptor. Thus, the physically detectable label allows the cells to be separated from a solution or retained in placed while a solution is removed. In a particular embodiment, the agent is a magnetic bead.

As discussed in relation to the first and second aspects, in an embodiment the agent is a bead, such as a magnetic bead, comprising immobilised anti-CD3 antibodies or anti-CD3 fragments thereof.

The enrichment comprises enriching the population for cells that are bound by the agent. The cells bound by the agent may be held in position while solution and other cells are removed. Alternatively, cells bound by the agent may be removed from the solution and other cells by physically removing the agent.

The transduced cells may be cultured under expansion conditions. The culturing under expansion conditions may be after culturing in the presence of feeder cells and may be after an enrichment step and/or enhancement step. The expansion conditions may comprise culturing the cells in the presence of one or more cytokines. For instance, the NK cells may be cultured in the presence of IL-2 and/or IL-15. In a particular embodiment, the NK cells are cultured in the presence of both IL-2 and IL-15. The IL-2 may be present at a concentration of at least 1, 10, 50, 100, 150, 175, 180, or 200 IU/ml. The IL-2 may be present at a concentration of 1-400 IU/ml, 50-350 IU/ml, 100-300 IU/ml, 150-250 IU/ml, 175-225 IU/ml, or approximately 200 IU/ml. The IL-2 may be present at a concentration of 1-400 IU/ml, 50-350 IU/ml, 100-300 IU/ml, 150-210 IU/ml, 170-190 IU/ml, or approximately 180 IU/ml. The IL-2 may be present at a concentration of 37-300 IU/ml, 110-260 IU/ml, 150-220 IU/ml, 160-190 IU/ml, 170-185 IU/ml, or approximately 180 IU/ml. The IL-2 may be present at a concentration of at least 0.14, 1.4, 6.8, 14, 20, 24, or 27 ng/ml. The IL-2 may be present at a concentration of 1-54 ng/ml, 6.8-47 ng/ml, 14-41 ng/ml, 20-34 ng/ml, 24-30 ng/ml, or approximately 27 ng/ml. The IL-2 may be present at a concentration of 0.14-54 ng/ml, 6.8-47 ng/ml, 14-41 ng/ml, 20-28 ng/ml, 23-26 ng/ml, or approximately 24 ng/ml. The IL-2 may be present at a concentration of 5-40 ng/ml, 15-35 ng/ml, 20-30 ng/ml, 22-26 ng/ml, 23-25 ng/ml, or approximately 24 ng/ml. The IL-15 may be present at a concentration of at least 0.1, 0.25, 0.5, 1, or 1.5 ng/ml. The IL-15 may be present at a concentration of 0.1-2 ng/ml, 0.25-1.75 ng/ml, 0.5-1.5 ng/ml, 0.75 to 1.25 ng/ml, or approximately 1 ng/ml. The IL-15 may be present at a concentration of 0.1-3 ng/ml, 1-2 ng/ml, 1.25-1.75 ng/ml, 1.4-1.6 ng/ml, or approximately 1.5 ng/ml. The IL-15 may be present at 0.5-2 ng/ml, 0.75-1.75 ng/ml, or 1-1.5 ng/ml. In a particular embodiment, culturing is in the presence of approximately 150-250 IU/ml IL-2 and approximately 0.5-1.5 ng/ml IL-15. In a particular embodiment, culturing is in the presence of approximately 150-210 IU/ml IL-2 and approximately 1.25-1.75 ng/ml IL-15. In another embodiment, culturing is in the presence of approximately 150-250 IU/ml IL-2 and approximately 0.75-1.75 ng/ml or 1-1.5 ng/ml IL-15. In a particular embodiment, culturing is in the presence of approximately 20-34 ng/ml IL-2 and approximately 0.5-1.5 ng/ml IL-15. In a particular embodiment, culturing is in the presence of approximately 20-30 ng/ml IL-2 and approximately 1.25-1.75 ng/ml IL-15. In another embodiment, culturing is in the presence of approximately 20-35 ng/ml IL-2 and 50 approximately 0.75-1.75 ng/ml or 1-1.5 ng/ml IL-15. In an embodiment, culturing is in the presence of approximately 24 ng/ml IL-2 and approximately 1.5 ng/ml IL-15. The cells may be cultured at 1×10⁵ to 1×10⁶ viable cells/ml, 1.5×10⁵ to 0.5×10⁵ viable cells/ml, 2×10⁵ to 3×10⁵ viable cells/ml, or approximately 2.5×10⁵ viable cells/ml.

As discussed herein, for the purposes of the present disclosure, IL-15 ng may be converted to IU/ml. The IL-15 concentrations in the preceding paragraph may be expressed as follows. The IL-15 may be present at a concentration of at least 1.2, 3, 6, 12, or 18 IU/ml. The IL-15 may be present at a concentration of 1.2-24 IU/ml, 3-21 IU/ml, 6-18 IU/ml, 9-15 IU/ml, or approximately 12 IU/ml. The IL-15 may be present at a concentration of 1.2-36 IU/ml, 12-24 IU/ml, 15-21 IU/ml, 17-19 IU/ml, or approximately 18 IU/ml. he IL-15 may be present at 6-24 IU/ml, 9-21 IU/ml, or 12-18 IU/ml. In a particular embodiment, culturing is in the presence of approximately 150-250 IU/ml IL-2 and approximately 6-18 IU/ml IL-15. In a particular embodiment, culturing is in the presence of approximately 150-210 IU/ml IL-2 and approximately 15-21 IU/ml IL-15. In another embodiment, culturing is in the presence of approximately 150-250 IU/ml IL-2 and approximately 9-21 IU/ml or 12-18 IU/ml IL-15. In a particular embodiment, culturing is in the presence of approximately 20-34 ng/ml IL-2 and approximately 6-18 IU/ml IL-15. In a particular embodiment, culturing is in the presence of approximately 20-30 ng/ml IL-2 and approximately 15-21 IU/ml IL-15. In another embodiment, culturing is in the presence of approximately 20-35 ng/ml IL-2 and approximately 9-21 IU/ml or 12-18 IU/ml IL-15. In an embodiment, culturing is in the presence of approximately 24 ng/ml IL-2 and approximately 18 IU/ml IL-15.

The cells may be expanded in any suitable culture vessel. For instance, the cells may be expanded in a bioreactor. Examples of bioreactors include a rocking bed bioreactor, a stirred tank bioreactor, a rotating wall bioreactor, a perfusion bioreactor, an isolation/expansion automated system, an automated or semi-automated bioreaction, a disposable bag bioreaction, and a gas permeable rapid expansion system. In particular, the bioreaction may be a gas permeable rapid expansion system, for instance the cells expanded using a G-Rex™ system. The cells may be expanded for, or for at least, 1, 2, 3, 4, 5, 6, 7, or 8 days. The cells may be expanded for, or for at least, 5, 6, 7, 8, 9, 10, 12, 14, or 16 days. In a particular embodiment, the cells are expanded for 8 days or 16 days. The cell culture media may be refreshed during the culture. As an example, the media may be refreshed every 3 days during the culturing under expansion conditions. For instance, the media may be refreshed 3 days and 6 days into the culturing under expansion conditions. The cells may be expanded for, or for at least, 10, 15, 20, 25, 30, 40, 50, 60, 70, 80, 90, or 100 days. Cytokines and/or feeder cells may be periodically refreshed during the culture. For instance, the cells may be expanded for 70 days and the culture may be pulsed five times with feeder cells. In an embodiment, the cells are expanded for 16 days and the cytokines are replenished 3 days into the expansion, the cytokines are replenished and feeder cells are added 6 days into the expansion, the medium is exchanged (e.g. 75% of the medium is exchanged) 9 days into the expansion, and/or the cytokines are replenished 12 days into the expansion.

After the expansion, the method may further comprise the cryopreservation of the TCR-NK cells as discussed herein. Alternatively, or subsequently, the method may comprise formulating the NK cells into a pharmaceutical composition, as discussed herein.

In a particular embodiment, there is provided a method of producing TCR-NK cells, the method comprising:
culturing NK cells in the presence of IL-2 and/or IL-15 and in the absence of feeder cells;
transducing the NK cells with one or more lentiviral vectors under conditions to transfer at least one nucleic acid encoding a TCR and one or more CD3 chains to the NK cells, wherein the transduction takes place on the 4^{th} day or 5^{th} day after the beginning of the culturing in the presence of IL-2 and/or IL-15; and
culturing the NK cells in the presence of feeder cells expressing mbIL-21 and 4-1BBL.

In a particular embodiment, there is provided a method of producing TCR-NK cells, the method comprising:
culturing NK cells in the presence of IL-2 and/or IL-15 and in the absence of feeder cells;
transducing the NK cells with one or more lentiviral vectors under conditions to transfer at least one nucleic acid encoding a TCR and one or more CD3 chains to the NK cells, wherein the transduction takes place on the 4^{th} day or 5^{th} day after the beginning of the culturing in the presence of IL-2 and/or IL-15;
transducing the NK cells with one or more lentiviral vectors under conditions to transfer at least one nucleic acid encoding a TCR and one or more CD3 chains to the NK cells, wherein the transduction takes place on the 6^{th} day or 7^{th} day after the beginning of the culturing in the presence of IL-2 and/or IL-15; and
culturing the NK cells in the presence of feeder cells expressing mbIL-21 and 4-1BBL.

In a particular embodiment, there is provided a method of producing TCR-NK cells, the method comprising:
culturing a population comprising NY cells in the presence of IL-2 and/or IL-15 and in the absence of feeder cells;
transducing the population of cells with one or more lentiviral vectors under conditions to transfer at least one nucleic acid encoding a TCR and one or more CD3 chains to the NK cells, wherein the transduction takes place on the 4^{th} day or 5^{th} day after the beginning of the culturing in the presence of IL-2 and/or IL-15; transducing the population of cells with one or more lentiviral vectors under conditions to transfer at least one nucleic acid encoding a TCR and one or more CD3 chains to the NK cells, wherein the transduction takes place on the 6^{th} day or 7^{th} day after the beginning of the culturing in the presence of IL-2 and/or IL-15;
culturing the post-transduction cells in the presence of feeder cells expressing mbIL-21 and 4-1BBL; and
contacting the population of cells with anti-CD3 molecules, optionally enriching for cells bound by the anti-CD3 molecules, and optionally resulting in a population that is enhanced for one or more function. The one or more function may be an increase in innate cytotoxicity and/or TCR-directed cytotoxicity.

In a particular embodiment, there is provided a method of producing TCR-NK cells, the method comprising:
culturing a population comprising NY cells in the presence of IL-2 and IL-15 and in the absence of feeder cells;
transducing the population of cells with one or more lentiviral vectors under conditions to transfer at least one nucleic acid encoding a TCR and one or more CD3 chains to the NK cells, wherein the transduction takes place on the 5^{th} day after the beginning of the culturing in the presence of IL-2 and IL-15;
transducing the population of cells with one or more lentiviral vectors under conditions to transfer at least one nucleic acid encoding a TCR and one or more CD3 chains to the NK cells, wherein the transduction takes place on the 7^{th} day after the beginning of the culturing in the presence of IL-2 and IL-15;
culturing the post-transduction cells in the presence of irradiated K562 cells expressing mbIL-21 and 4-1BBL; and
culturing the enriched population of cells in the presence of IL-2 and IL-15 in a gas permeable rapid expansion system.

In a particular embodiment, there is provided a method of producing TCR-NK cells, the method comprising:
culturing a population comprising NY cells in the presence of IL-2 and/or IL-15 and in the absence of feeder cells;
transducing the population of cells with one or more lentiviral vectors under conditions to transfer at least one nucleic acid encoding a TCR and one or more CD3 chains to the NK cells, wherein the transduction takes place on the 4^{th} day or 5^{th} day after the beginning of the culturing in the presence of IL-2 and/or IL-15;
transducing the population of cells with one or more lentiviral vectors under conditions to transfer at least one nucleic acid encoding a TCR and one or more CD3 chains to the NK cells, wherein the transduction takes place on the 6^{th} day or 7^{th} day after the beginning of the culturing in the presence of IL-2 and/or IL-15;
culturing the post-transduction cells in the presence of feeder cells expressing mbIL-21 and 4-1BBL;
contacting the population of cells with anti-CD3 molecules, optionally enriching for cells bound by the anti-CD3 molecules, and optionally resulting in a population that is enhanced for one or more function, such as increasing in innate cytotoxicity and/or TCR-directed cytotoxicity; and
culturing the enriched population of cells in the presence of IL-2 and/or IL-15, optionally in a gas permeable rapid expansion system.

In a particular embodiment, there is provided a method of producing TCR-NK cells, the method comprising:
culturing a population comprising NYK cells in the presence of IL-2 and IL-15 and in the absence of feeder cells;
transducing the population of cells with one or more lentiviral vectors under conditions to transfer at least one nucleic acid encoding a TCR and one or more CD3 chains to the NK cells, wherein the transduction takes place on the 5^{th} day after the beginning of the culturing in the presence of IL-2 and IL-15;
transducing the population of cells with one or more lentiviral vectors under conditions to transfer at least one nucleic acid encoding a TCR and one or more CD3 chains to the NK cells, wherein the transduction takes place on the 7^{th} day after the beginning of the culturing in the presence of IL-2 and IL-15;
culturing the post-transduction cells in the presence of irradiated K562 cells expressing mbIL-21 and 4-1BBL;
contacting the population of cells with anti-CD3 molecules and enriching for cells bound by the anti-CD3 molecules; and
culturing the enriched population of cells in the presence of IL-2 and IL-15 in a gas permeable rapid expansion system.

In a particular embodiment, there is provided a method of producing TCR-NK cells, the method comprising:
culturing a population comprising NYK cells in the presence of 50-80 ng/ml IL-2 and 0.5-10 ng/ml IL-15 and in the absence of feeder cells;
transducing the population of cells with one or more lentiviral vectors under conditions to transfer at least one nucleic acid encoding a TCR and one or more CD3 chains to the NK cells, wherein the transduction takes place on the 5^{th} day after the beginning of the culturing in the presence of IL-2 and IL-15;
transducing the population of cells with one or more lentiviral vectors under conditions to transfer at least one nucleic acid encoding a TCR and one or more CD3 chains to the NK cells, wherein the transduction takes place on the 7^{th} day after the beginning of the culturing in the presence of IL-2 and IL-15;
culturing the post-transduction cells in the presence of irradiated K562 cells expressing mbIL-21 and 4-1BBL, optionally at a K562:NK cell ratio of 2:1, and optionally in the presence of 20-35 ng/ml IL-2 and 0.5-2 ng/ml IL-15;
contacting the population of cells with anti-CD3 molecules and enriching for cells bound by the anti-CD3 molecules; and
culturing the enriched population of cells in the presence of 20-35 ng/ml IL-2 and 0.5-2 ng/ml IL-15 in a gas permeable rapid expansion system and culturing the enriched population of cells in the presence of irradiated K562 cells expressing mbIL-21 and 4-1BBL, optionally at a K562:NK cell ratio of 2:1, in the gas permeable rapid expansion system.

In a particular embodiment, there is provided a method of producing TCR-NK cells, the method comprising:
i) purifying NK cells at day 0 and culturing the NK cells in the presence of 50-80 ng/ml IL-2 (e.g. 60 ng/ml IL-2) and 0.5-10 ng/ml IL-15 (e.g. 1 ng/ml or 7 ng/ml IL-15) and in the absence of feeder cells;
ii) at day 5 transducing the population of cells with one or more lentiviral vectors under conditions to transfer at least one nucleic acid encoding a TCR and one or more CD3 chains to the NK cells;
iii) at day 7 transducing the population of cells with one or more lentiviral vectors under conditions to transfer at least one nucleic acid encoding a TCR and one or more CD3 chains to the NK cells;
iv) at day 8 culturing the post-transduction cells in the presence of irradiated K562 cells expressing mbIL-21 and 4-1BBL, optionally at a K562:NK cell ratio of 2:1, and in the presence of 20-35 ng/ml IL-2 and 0.5-2 ng/ml IL-15;
v) at day 12 contacting the population of cells with anti-CD3 molecules and enriching for cells bound by the anti-CD3 molecules and culturing the enriched population of cells in the presence of 20-35 ng/ml IL-2 and 0.5-2 ng/ml IL-15;
vi) replenishing the 20-35 ng/ml IL-2 and 0.5-2 ng/ml IL-15;
vii) at day 18 adding irradiated K562 cells expressing mbIL-21 and 4-1BBL, optionally at a K562:NK cell ratio of 2:1, and in the presence of 20-35 ng/ml IL-2 and 0.5-2 ng/ml IL-15;
viii) exchanging the media (e.g. exchanging 75% of the media);
ix) replenishing the 20-35 ng/ml IL-2 and 0.5-2 ng/ml IL-15; and
x) at day 28 cryopreserving the cells.

In a particular embodiment, there is provided a method of producing TCR-NK cells, the method comprising:
i) purifying NK cells at day 0 and culturing the NK cells in the presence of 50-80 ng/ml IL-2 (e.g. 60 ng/ml IL-2) and 0.5-10 ng/ml IL-15 (e.g. 7 ng/ml IL-15) and in the absence of feeder cells;
ii) at day 5 transducing the population of cells with one or more lentiviral vectors under conditions to transfer at least one nucleic acid encoding a TCR and one or more CD3 chains to the NK cells;
iii) at day 7 transducing the population of cells with one or more lentiviral vectors under conditions to transfer at least one nucleic acid encoding a TCR and one or more CD3 chains to the NK cells;
iv) at day 8 culturing the post-transduction cells in the presence of irradiated K562 cells expressing mbIL-21 and 4-1BBL, at a K562:NK cell ratio of 2:1, and in the presence of 20-35 ng/ml IL-2 and 0.5-2 ng/ml IL-15;
v) at day 12 contacting the population of cells with anti-CD3 molecules and enriching for cells bound by the anti-CD3 molecules and culturing the enriched population of cells in the presence of 20-35 ng/ml IL-2 and 0.5-2 ng/ml IL-15;
vi) at day 15 replenishing the 20-35 ng/ml IL-2 and 0.5-2 ng/ml IL-15;
vii) at day 18 adding irradiated K562 cells expressing mbIL-21 and 4-1BBL, optionally at a K562:NK cell ratio of 2:1, and in the presence of 20-35 ng/ml IL-2 and 0.5-2 ng/ml IL-15;
viii) at day 21 exchanging the media (e.g. exchanging 75% of the media);
ix) at day 24 replenishing the 20-35 ng/ml IL-2 and 0.5-2 ng/ml IL-15; and
x) at day 28 cryopreserving the cells.

In a seventh aspect, there is provided a population of or comprising transduced NK cells that is obtained or obtainable by any of the methods of the sixth aspect of the present disclosure.

In an eighth aspect, there is provided NY cells of the seventh aspect for use in a method of treatment. The method of treatment may be as discussed in relation to the fourth or fifth aspects of the present disclosure. In particular, the subjects to be treated, nature of the treatments, pathologies, methods of administration, treatment regimens, etc may be as discussed in relation to the fourth or fifth aspects.

All of the features described herein (including any accompanying claims, abstract and drawings), and/or all of the steps of any method or process so disclosed, may be combined with any of the above aspects in any combination, except combinations where at least some of such features and/or steps are mutually exclusive.

Cytokine concentrations provided herein may be the concentration at the start of a culture. For prolonged incubations, the culture media may, in some examples, be refreshed periodically and the cytokine concentration may be restored to the starting concentration.

For a better understanding of the invention, and to show how embodiments of the same may be carried into effect, reference will now be made to the Examples, which are not intended to limit the invention in any way.

### EXAMPLES

### Example 1—Evaluation of Sorting Strategies: ‘Post-Transduction Enrichment’

TCR-NK cells were generated by lentiviral transduction after pre-activation in the presence of IL-2 and IL-15 and the absence of feeder cells, as disclosed herein (see Example 8). The TCR-NK cells were then sorted using CD3 targeting beads (Miltenyi) or TCR targeting beads (Mitenyi).

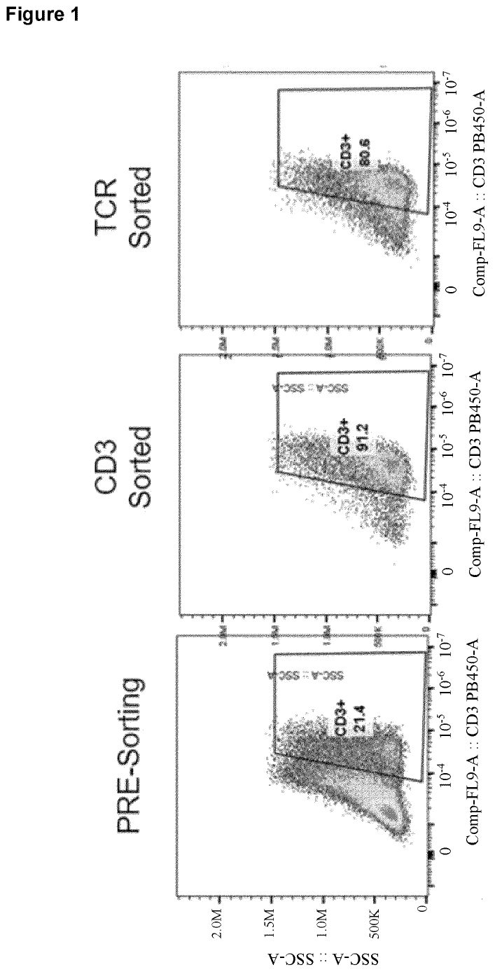

As illustrated in FIG. 1, the anti-CD3 beads achieved a higher level of recovery (80-96%) compared to the anti-TCR beads. This was tested for three separate donors and the data from the highest transduction is in FIG. 1.

The cells by further analysed to determine the expression of various proteins and the results are presented in the table below.

| Marker | % positive cells (CD3 sort) |
|---|---|
| CD3 | >68% (91% for depicted donor) |
| TCR | >24% (77% for depicted donor) |
| Dextramer | >37% (73% for depicted donor) |
| CD56 | >90% |
| CD16 | >60% |
| NKG2D | >95% |
| NKG2C | Variable (between donors) |
| NKp30/46 | Variable (between donors) |
| NKp44 | Variable (between donors) |
| TRAIL | Variable (between donors) |
| FasL | Variable |
| NKG2A | Variable (between donors) |

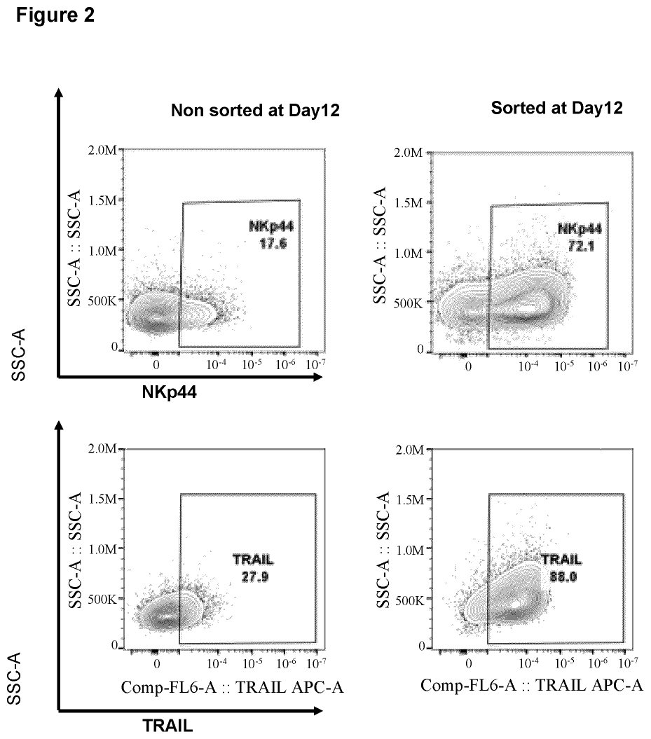

FIG. 2 illustrates the effect on NKp44 and TRAIL expression from an exemplary experiment. Sorting with anti-CD3 beads increased the expression of these markers.

### Example 2—Implementation of a Post-Transduction Sorting Step Ensuring Consistent % TCR⁺

TCR-NK cells were generated by lentiviral transduction after pre-activation in the presence of IL-2 and IL-15 and the absence of feeder cells, as disclosed herein (see Example 8). At day 12 post-thaw, the TCR-NK cells were then sorted using magnetic CD3 targeting beads (Miltenyi).

| | | % CD3 | % CD3 |
|---|---|---|---|
| Run/Donor | TCR | Non-Sorted | Sorted |
| Run197, BD009 | TCR#1 | 23 | 80 |
| Run197, BD090 | TCR#1 | 19 | 68 |
| Run197, BD098 | TCR#1 | 30 | 91 |
| Run214, Donor1 | TCR#1 | 12 | 69 |
| Run214, Donor1 | TCR#2 | 11 | 81 |
| Run214, Donor2 | TCR#1 | 78 | 99 |
| Run214, Donor2 | TCR#2 | 79 | — |
| Run214, Donor3 | TCR#1 | 23 | 91 |
| Run214, Donor3 | TCR#2 | 26 | 85 |
| Run214, Mix donor | TCR#1 | 23 | 86 |
| Run214, Mix donor | TCR#2 | 23 | 77 |

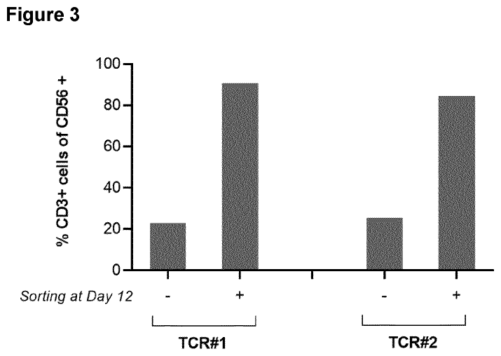

Post-transduction sorting was performed ten times (2 runs): 7× with TCR #1 and 3× with TCR #2. The percentage of transduced post-sort at day 20 ranges from 68% to 99% CD3+. Representative data (pre/post sort) from donor #3 Run214 are illustrated in FIG. 3.

### Example 3—TCR-NK Sorted Product has Compelling Killing Performance

TCR-NK cells were generated by lentiviral transduction after pre-activation in the presence of IL-2 and IL-15 and the absence of feeder cells, as disclosed herein (see Example 8). At day 12 post-thaw, the TCR-NK cells were then sorted using magnetic CD3 targeting beads (Miltenyi). Control cells were not sorted. From days 12 to 20 the cells were expanded in G-Rex™. At day 20 the product was evaluated for killing performance.

The post transduction-sorting process was tested six times (2 run, 6 donors). The killing assay included an Effector:Target ratio (E:T) of 4:1. The co-incubation was for 4 hours. Higher killing was observed.

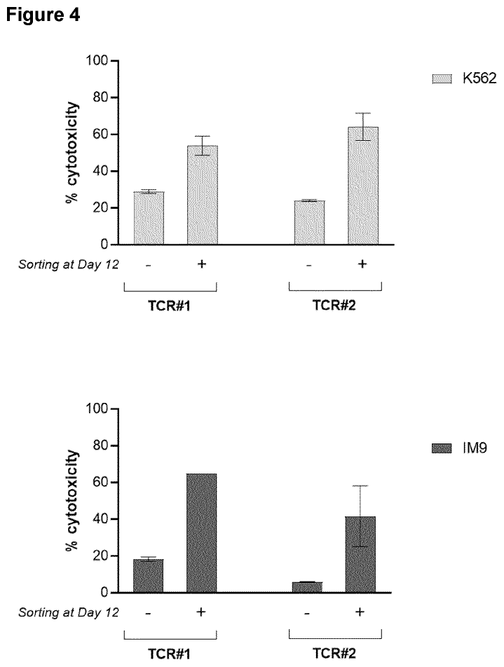

FIG. 4 illustrates representative data from Donor #3 (Run214).

Two types of target cell were tested. One target was K562 which is a chronic myelogenous leukaemia cell line and is MAGE-A4⁻ (upper panel of FIG. 4). This target is to test innate killing. Another target was IM9 which is a multiple myeloma cell line and is HLA-A2+ and MAGE-A4+(lower panel of FIG. 4). This target is to test TCR-directed killing.

Both higher innate killing and higher TCR-directed killing was observed for cells that were sorted using anti-CD3 beads.

### Example 4—TCR-NK Sorted Product has Improved Degranulation

TCR-NK cells were generated by lentiviral transduction after pre-activation in the presence of IL-2 and IL-15 and the absence of feeder cells, as disclosed herein (see Example 8). At day 12 post-thaw, the TCR-NK cells were then sorted using magnetic CD3 targeting beads (Miltenyi). Control cells were not sorted. From days 12 to 20 the cells were expanded in G-Rex™. At day 20 the product was evaluated for degranulation performance.

A degranulation assay was carried out using NCI-H1703 (a non-small lung cancer epithelial cell line that is HLA2⁺ and MAGE-A4⁺) as a target.

The assay was run for three donors (1 run), the E:T was 1:2, and the co-incubation was for 5 hours. FIG. 5 shows representative data from Donor #3.

An increase in degranulation was observed for cells that were sorted using anti-CD3 beads.

### Example 5—Cell Expansion in Bioreactor

TCR-NK cells were generated by lentiviral transduction after pre-activation in the presence of IL-2 and IL-15 and the absence of feeder cells, as disclosed herein (see Example 8). At day 12 post-thaw, the TCR-NK cells were then sorted using magnetic CD3 targeting beads (Miltenyi). Control cells were not sorted. From days 12 to 20 the cells were expanded in G-Rex™.

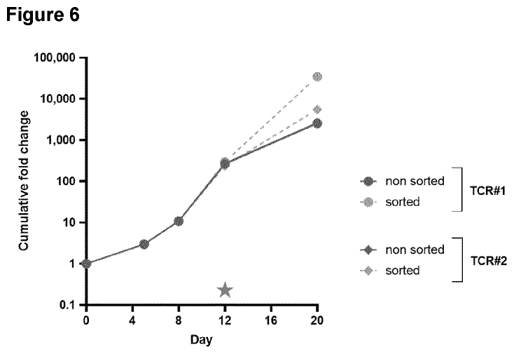

A similar proliferative capacity was observed with or without sorting. Therefore, the anti-CD3 sorting does not negatively affect expansion. Illustrative results are presented in FIG. 6.

### Example 6—Transduction Window—Single Transduction

NK cells were thawed, selected, and pre-activated in the presence of IL-2 and IL-15 and in the absence of feeder cells, as discussed for Example 8. The day of thawing and the start of pre-activation is considered “day 0”.

The pre-activated NK cells were transduced on day 2, day 3, day 4, or day 5. The best transduction window was found to be between day 4 and day 5.

The above experiment was performed for cells from three donors. The cells were transduced with the TCR “TCR #1” and all four CD3 chains. Variability was seen between donors but the kinetic trends were similar.

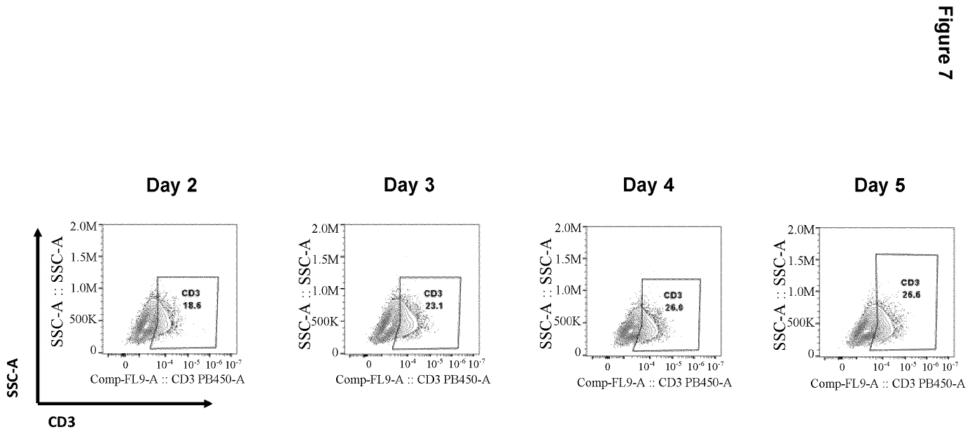

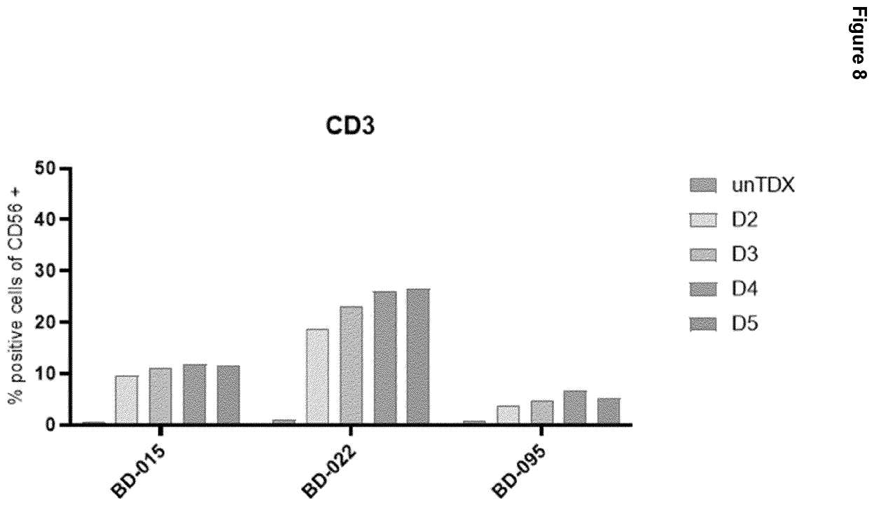

Illustrative plots are presented in FIG. 7 and overall results are presented in FIG. 8.

### Example 7—Evaluation of Transduction: ‘TDX Window’, ‘Single Vs Double’, ‘O/N TDX’

NK cells were thawed, selected, and pre-activated in the presence of IL-2 and IL-15 and in the absence of feeder cells, as discussed for Example 8. The day of thawing and the start of pre-activation is considered “day 0”.

The cells were singly transduced on day 4, singly transduced overnight on day 4 (for 18 hours), transduced on day 4 and again on day 6, or transduced on day 5 and again on day 7.

The above experiment was performed for cells from three donors. The cells were transduced with the TCR “TCR #1” and all four CD3 chains. Variability was seen between donors but the kinetic trends were similar.

The double transduction was improved over the single transduction. The cells had a better phenotype and similar functionality. The best transduction was the double transduction at days 5 and 7, and this result was seen for all three donors and for both CD3 expression and TCR expression.

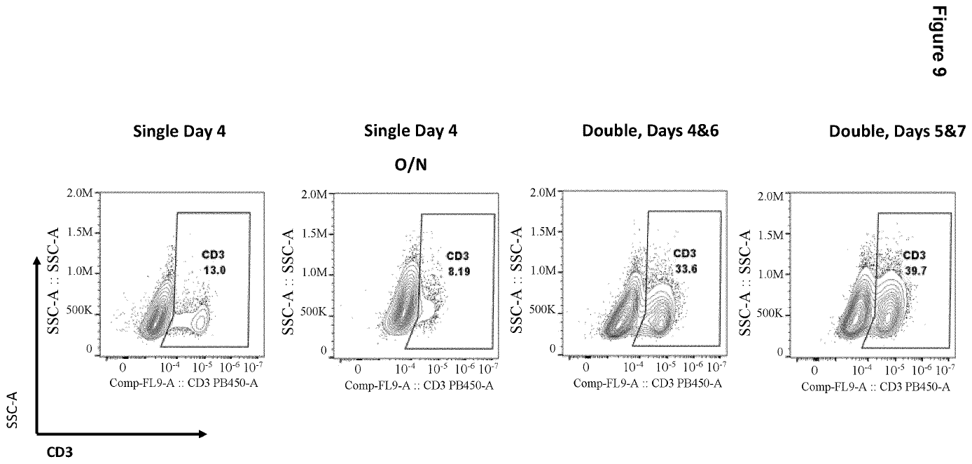

Illustrative results are presented in FIG. 9.

The experiment was repeated to test the double transduction at days 5 and 7 for other TCRs. Cells from three donors were tested.

### Example 8—Exemplary Method of Making TCR-NK Cells Suitable for Therapeutic Purposes

An exemplary method for making TCR-NK cells comprises: Day 0, thawing & CD3⁻CD56⁺ magnetic selection; Day 5 & Day 7, dual viral transduction (by spinoculation with enhancers); Day 8, irradiated Feeder-pulsing; Day 12, CD3⁺ magnetic enrichment; Day 12 to Day 20, cell expansion in GRex; and Day 20, formulation, F&F and cryopreservation. Illustrative conditions are listed below.

An apheresis was thawed, washed, and NK cells were purified by CD3 negative selection (CD3 Microbeads, Miltenyi) followed by CD56 positive selection (CD56 Microbeads, Miltenyi) according to the manufacturer's instructions. The cells were suspended in a NK culture medium consisting in NK MACS (Miltenyi) supplemented with human serum 5%, IL-2 (500 IU/ml) and IL-15 (1 ng/ml) at a density of 1×10⁶ viable cells/mL and cultured in T-flasks.

On day 5, the cells were transduced with a mix of two separate VSV-g pseudotyped lentiviral vectors, carrying a T-cell receptor (TCR) and the CD3 molecule (with all four CD3 chains linked together using 2A peptides). Briefly, the cells were suspended at a cell density of 1×10⁶ cells/mL in the NK culture medium in the presence of the two lentiviral vectors (at a multiplicity of infection (MOI) of 20), protamine sulfate (10 μg/mL) and BX795 (5 μM). Transduction was done under dynamic conditions, by spinoculation (900 g, 60 min at 32° C.), followed by static incubation for 5 hours. After that, the cells were resuspended in a NK MACS (Miltenyi) supplemented with human serum 5%, IL-2 (200 IU/ml) and IL-15 (1 ng/ml) at a density of 1×10⁶ viable cells/mL.

On day 7, the cells were transduced a second time, as described above.

On day 8, the cells were suspended in fresh NK culture medium and seeded in 6-well G-rex plates at a cell density of 0.25×10⁶ viable cells/mL. Irradiated K562 feeder cells expressing membrane-bound IL-21 and 4-1BB ligand were added to the culture, at a NK to feeder ratio of 1:2.

On day 12, the TCR-NK cells were enriched by CD3 positive selection (CD3 Microbeads, Miltenyi) according to the manufacturer's instructions. The cells were then suspended in a NK culture medium consisting in NK MACS (Miltenyi) supplemented with human serum 5%, IL-2 (200 IU/ml) and IL-15 (1 ng/ml) at a density of 0.25×10⁶ viable cells/mL and cultured in 6-well G-rex plates.

On day 15 and day 18, 75% of the NK culture medium was refreshed.

On day 20, the cells were harvested and used fresh, or were frozen down in a custom cryoformulation comprising 5% DMSO.

### Example 9—Exemplary Method of Making TCR-NK Cells Suitable for Therapeutic Purposes

Below is an exemplary method that was tested for the generation of TCR-NK cells. In the below, “LV” refers to a lentiviral vector and “KVL” refers to a specific TCR.

| Day | Process Step | Exemplary Process Parameter |
|---|---|---|
| 0 | NK cells purification | Cell seeding density of [0.5-1.0] × 10⁶ |
| | | cells/cm² |
| | | IL-2 concentration of 60 ng/mL |
| | | IL-15 concentration of 7 ng/mL |
| | ↓ | |
| 5, 7 | Transduction | LV-CD3-a MOI [15-25] IU/cell |
| | | LV-KVL-a MOI [10-20] IU/cell |
| | | Protamine Sulfate concentration of 10 |
| | | μg/mL |
| | | BX795 concentration of 5 μM |
| | ↓ | |
| 8 | Irradiated feeder pulse | Feeder to cell ratio of 2:1 |
| | | Cell seeding density of [0.125-0.25] × |
| | | 10⁶ cell/cm² |
| | | IL-2 concentration of 24 ng/mL |
| | | IL-15 concentration of 1.5 ng/mL |
| | ↓ | |
| 12 | TCR-NK enrichment | Cell seeding density of [0.125-0.25] × |
| | (anti-CD3 based) | 10⁶ cell/cm² |
| | | IL-2 concentration of 24 ng/mL |
| | | IL-15 concentration of 1.5 ng/mL |
| | ↓ | |
| 15 | Cytokine | IL-2 concentration of 24 ng/mL |
| | replenishment | IL-15 concentration of 1.5 ng/mL |
| | ↓ | |
| 18 | Irradiated feeder pulse | Feeder to cell ratio of 2:1 |
| | | IL-2 concentration of 24 ng/mL |
| | | IL-15 concentration of 1.5 ng/mL |
| | ↓ | |
| 21 | Medium exchange | 75% medium exchange |
| | ↓ | |
| 24 | Cytokine replenishment | IL-2 concentration of 24 ng/mL |
| | | IL-15 concentration of 1.5 ng/mL |
| | ↓ | |
| 28 | Wash, formulation and | Use relevant cryoformulation and freeze |
| | Cryopreservation | |

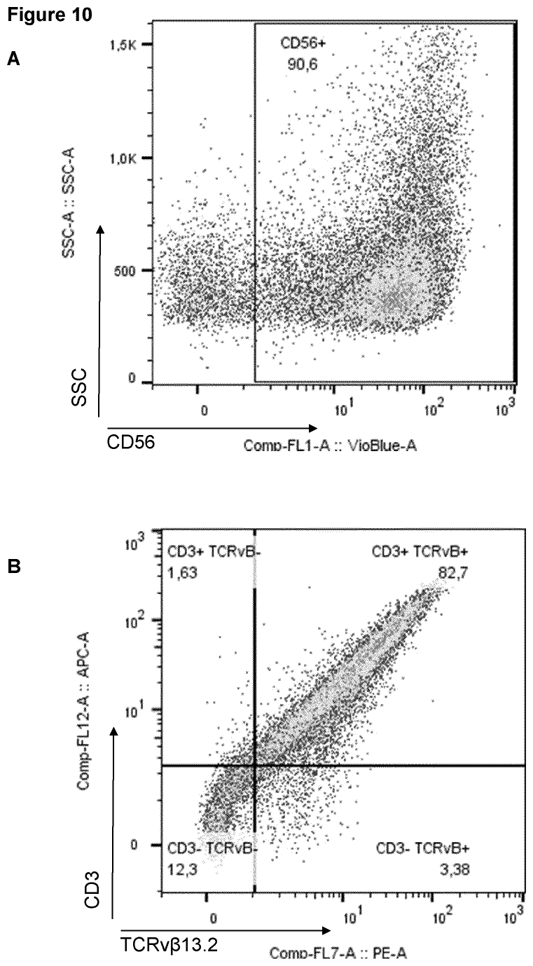

FIG. 10 shows the cellular output generated using the above process. In FIG. 10A, it can be seen that approximately 91% of the output cells express CD56. FIG. 10B shows the percentage of CD3+ and TCR+ cells in the product at the end of the process; approximately 83% of the cells express the TCR-CD3 complex.

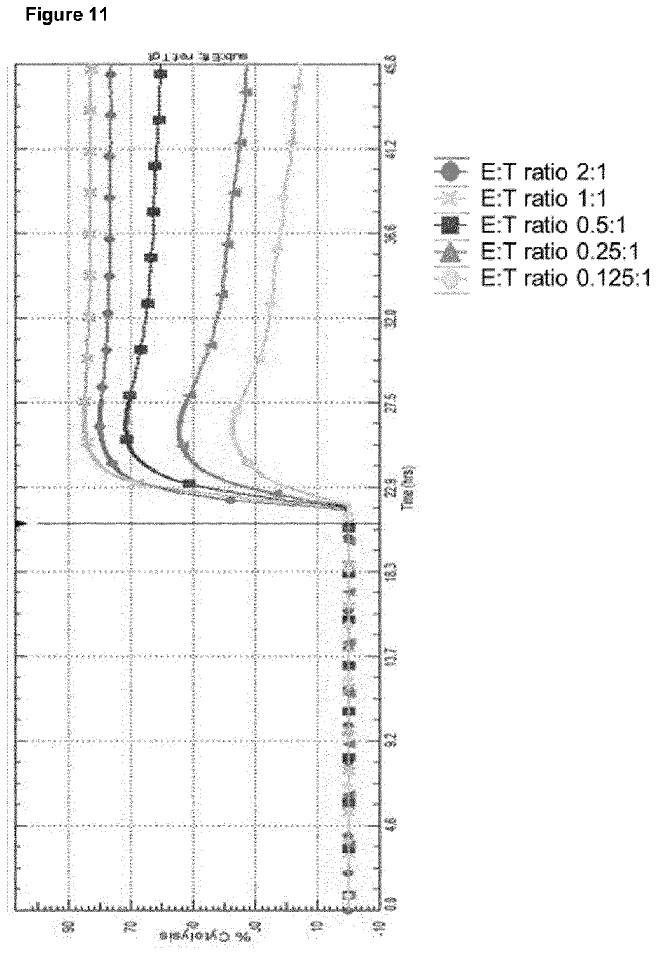

FIG. 11 shows the potency of the TCR-NK cells generated using the described process. TCR-NK cells expressing a TCR recognizing a peptide derived from the cancer testis antigen MAGE-A4 in the context of HLA-A2 were incubated with the cancer cell line NCI-H1703 which express both the MAGE-A4 protein and HLA-A2. Cytotoxicity was measured at different effector:target ratios using Incucyte.

1. A method of enriching natural killer (NK) cells that express a CD3-TCR complex (TCR-NK cells) within a population of cells, the method comprising:
obtaining a population of cells comprising TCR-NK cells; and
enriching for TCR-NK cells using surface CD3 as a marker.

2. The method of claim 1, wherein the method comprises contacting the population of cells with anti-CD3 antibodies or anti-CD3 fragments thereof.

3. The method of claim 2, wherein the anti-CD3 antibodies or anti-CD3 fragments thereof are displayed on a substrate.

4. The method of claim 3, wherein the substrate is a nanomatrix or a bead.

5. The method of any preceding claim, wherein the enriching comprises contacting the population of cells with a substrate displaying anti-CD3 antibodies or anti-CD3 fragments thereof and retaining cells bound by said substrate.

6. The method of any preceding claim, wherein the enriching comprises contacting the population of cells with magnetic beads displaying anti-CD3 antibodies or anti-CD3 fragments thereof and using magnetism to retain cells bound by said beads.

7. A method for enhancing one or more functions of a population of cells comprising TCR-NK cells, the method comprising:
obtaining a population of cells comprising TCR-NK cells; and
contacting the population of cells with an agent capable of binding to CD3.

8. The method of claim 7, wherein the one or more functions includes innate cytotoxicity and/or TCR-directed cytotoxicity.

9. The method of claim 7 or claim 8, wherein the enhanced population has increased innate cytotoxicity or TCR-directed cytotoxicity compared to a control population that was not contacted with an agent capable of binding to CD3.

10. The method of any one of claims 7 to 9, wherein the agent comprises anti-CD3 antibodies or anti-CD3 fragments thereof.

11. The method of any one of claims 7 to 10, wherein the agent comprises a substrate displaying the anti-CD3 antibodies or anti-CD3 fragments thereof.

12. The method of claim 11, wherein the substrate is a bead or a nanomatrix.

13. The method of any one of claims 7 to 12, wherein the method comprises a step of enriching the population for cells that are bound by the agent.

14. The method of any preceding claim, wherein the population of cells comprising TCR-NK cells is obtained by:
culturing a population comprising NK cells under activating conditions; and
transducing the activated NK cells under conditions to transfer at least one exogenous nucleic acid to the NK cells, wherein the one or more exogenous nucleic acids encode a TCR and/or one or more CD3 chains.

15. The method of claim 14, wherein the transduction comprises the use of one or more viral vectors.

16. The method of claim 14 or claim 15, wherein the population of cells is cultured in the presence of feeder cells after the transduction.

17. The method of claim 16, wherein the feeder cells are K562 cells that express mbIL-21 and 4-1BBL.

18. The method of any preceding claim, wherein the population of cells is cultured under expansion conditions after being enriched and/or enhanced.

19. The method of claim 18, wherein the expansion conditions are:
in presence of IL-2 and/or IL-15; and/or
for at least 1, 2, 3, 4, 5, 6, 7, or 8 days or for at least 5, 6, 7, 8, 9, 10, 12, 14, or 16 days.

20. The method of any preceding claim, wherein the enriched/enhanced population is suitable for therapeutic infusion.

21. The method of any preceding claim, wherein the method further comprises cryopreserving the enriched/enhanced population and/or formulating the enriched/enhanced population into a pharmaceutical composition.

22. A method of producing natural killer (NK) cells that comprise one or more exogenous nucleic acids encoding a TCR or portion thereof, the method comprising:
i) culturing NK cells under activating conditions; and
ii) transducing the activated NK cells under conditions to transfer the one or more exogenous nucleic acids to the NK cells, wherein the transduction takes place on the 4th day or later day after the beginning of step i).

23. The method of claim 22, wherein the transduction takes place:
more than 80 hours, more than 88 hours, more than 96 hours, more than 112 hours, more than 120 hours, more than 128 hours, or more than 136 hours after the beginning of step i);
80 to 143 hours, 96 to 136 hours, or 112 to 130 hours after the beginning of step i);
on the 4^{th} or 5^{th} day after the beginning of step i); or
on the 5^{th} day after the beginning of step i).

24. The method of claim 22 or 23, wherein the transduction is a first transduction and the method further comprises a second transduction on the 6^{th} day or later day after the beginning of step i).

25. The method of claim 24, wherein the second transduction takes place:
more than 128 hours, more than 136 hours, more than 144 hours, more than 160 hours, more than 168 hours, more than 176 hours, or more than 184 hours after the beginning of step i);
128 to 191 hours, 144 to 184 hours, or 160 to 178 hours after the beginning of step i);
on the 6^{th} or 7^{th} day after the beginning of step i); or
on the 7^{th} day after the beginning of step i).

26. The method of any one of claims 22 to 25, wherein the one or more exogenous nucleic acids encode a TCR-CAR.

27. The method of any one of claims 22 to 25, wherein the one or more exogenous nucleic acids encode a TCR and/or one or more CD3 chains, and lead to the generation of TCR-NK cells.

28. The method of claim 24 or claim 25, wherein the first and/or second transduction comprises the transduction of the NK cells with a TCR and one or more CD3 chains, and lead to the generation of TCR-NK cells.

29. The method of any one of claims 22 to 28, wherein the transduction comprises the use of one or more lentiviral vectors.

30. The method of any one of claims 22 to 29, wherein the transduction is in the presence of BX795 and/or protamine sulfate.

31. The method of any one of claims 22 to 30, wherein step i) comprises culturing the NK cells in the presence of one or more cytokines.

32. The method of any one of claims 22 to 31, wherein step i) comprises culturing the NK cells in the presence of IL-2 and/or IL-15.

33. The method of claim 32, wherein:
the IL-2 is present at a concentration of:
1-1000 IU/ml, 10-900 IU/ml, 100-800 IU/ml, 250-750 IU/ml, 400-600 IU/ml, 450-550 IU/ml, or approximately 500 IU/ml; or
220-670 IU/ml, 330-560 IU/ml, 370-520 IU/ml, 410-480 IU/ml, or approximately 440 IU/ml; or
0.14-140 ng/ml, 1.4-120 ng/ml, 14-110 ng/ml, 34-100 ng/ml, 54-81 ng/ml, 61-74 ng/ml, or approximately 68 ng/ml; or
30-90 ng/ml, 45-75 ng/ml, 50-70 ng/ml, 55-65 ng/ml, or approximately 60 ng/ml; or
50-80 ng/ml or 60-70 ng/ml; and/or
the IL-15 is present at a concentration of:
0.1-2 ng/ml, 0.25-1.75 ng/ml, 0.5-1.5 ng/ml, 0.75 to 1.25 ng/ml, or approximately 1 ng/ml; or
2-12 ng/ml, 3-11 ng/ml, 4-10 ng/ml, 5-9 ng/ml, 6-8 ng/ml, or approximately 7 ng/ml; or
0.5-10, 0.7-9, 0.9-8, or 1-7 ng/ml; optionally
wherein the IL-2 is present at a concentration of 50-80 ng/ml or 60-70 ng/ml and the IL-15 is present at a concentration of 0.5-10, 0.7-9, 0.9-8, or 1-7 ng/ml.

34. The method of any one of claims 22 to 33, wherein step i) is in the absence of feeder cells.

35. The method of any one of claims 22 to 33, wherein, after step ii), the method comprises culturing the cells in the presence of feeder cells.

36. The method of claim 35, wherein the feeder cells are K562 cells.

37. The method of claim 35 or claim 36, wherein the feeder cells express membrane-bound IL-21 (mbIL-21) and 4-1BBL.

38. The method of any one of claims 22 to 37, comprising enriching NK cells that have been transduced using surface CD3 or TCR as a marker.

39. The method of any one of claims 22 to 38, comprising contacting the NK cells with an anti-CD3 molecule.

40. The method of claim 38 or 39, wherein the NK cells are contacted with beads displaying anti-CD3 antibodies or anti-CD3 fragments thereof.

41. The method of any one of claims 38 to 40, wherein the enrichment step of claim 38 and/or the contacting step of claim 39 or claim 40 takes place after the culturing step of claims 35 to 37.

42. The method of any one of claims 22 to 41, wherein, after step ii), the method comprises culturing the NK cells under expansion conditions in the presence of one or more cytokines.

43. The method of claim 42, wherein the culturing the NK cells under expansion conditions takes place after the enrichment and/or contacting step of claims 38 to 41.

44. The method of claim 42 or claim 43, wherein the one or more cytokines are IL-2 and/or IL-15.

45. The method of claim 44, wherein:
the IL-2 is present at a concentration of:
1-400 IU/ml, 50-350 IU/ml, 100-300 IU/ml, 150-250 IU/ml, 175-225 IU/ml, or approximately 200 IU/ml; or
1-400 IU/ml, 50-350 IU/ml, 100-300 IU/ml, 150-210 IU/ml, 170-190 IU/ml, or approximately 180 IU/ml; or
37-300 IU/ml, 110-260 IU/ml, 150-220 IU/ml, 160-190 IU/ml, 170-185 IU/ml, or approximately 180 IU/ml; or
1-54 ng/ml, 6.8-47 ng/ml, 14-41 ng/ml, 20-34 ng/ml, 24-30 ng/ml, or approximately 27 ng/ml; or
0.14-54 ng/ml, 6.8-47 ng/ml, 14-41 ng/ml, 20-28 ng/ml, 23-26 ng/ml, or approximately 24 ng/ml; or
5-40 ng/ml, 15-35 ng/ml, 20-30 ng/ml, 22-26 ng/ml, 23-25 ng/ml, or approximately 24 ng/ml; or
20-35 ng/ml; and/or
the IL-15 is present at a concentration of:
0.1-2 ng/ml, 0.25-1.75 ng/ml, 0.5-1.5 ng/ml, 0.75 to 1.25 ng/ml, or approximately 1 ng/ml; or
0.1-3 ng/ml, 1-2 ng/ml, 1.25-1.75 ng/ml, 1.4-1.6 ng/ml, or approximately 1.5 ng/ml; or
0.5-2 ng/ml, 0.75-1.75 ng/ml, or 1-1.5 ng/ml; optionally
wherein the IL-2 is present at a concentration of 20-35 ng/ml and the IL-15 is present at a concentration of 0.75-1.75 ng/ml or 1-1.5 ng/ml.

46. The method of any one of claims 35 to 45, wherein the culturing in the presence of feeder cells and/or the culturing under expansion conditions comprises use of a gas permeable rapid expansion system.

47. The method of any one of claims 22 to 46, wherein the method further comprises cryopreserving the NK cells and/or formulating the NK cells into a pharmaceutical composition.

48. The method of any one of claims 22 to 47, wherein
step i) comprises culturing NK cells in the presence of IL-2 and/or IL-15 and in the absence of feeder cells;
step ii) comprises transducing the NK cells with one or more lentiviral vectors under conditions to transfer at least one nucleic acid encoding a TCR and one or more CD3 chains to the NK cells, wherein the transduction takes place on the 4^{th} or 5^{th} day after the beginning of step i); and
step ii) comprises transducing the NK cells with one or more lentiviral vectors under conditions to transfer at least one nucleic acid encoding a TCR and one or more CD3 chains to the NK cells, wherein the transduction takes place on the 6^{th} or 7^{th} day after the beginning of step i); and
after the transduction the method comprises culturing the NK cells in the presence of feeder cells expressing mbIL-21 and 4-1BBL.

49. An NK cell obtained or obtainable by any one of the methods of claims 1 to 48.

50. A TCR-NK cell obtained or obtainable by any one of the methods of claims 1 to 48.

51. A population comprising NK cells for use in a method of treatment, wherein the population has been generated by a method according to any one of claims 1 to 48.

52. A population comprising TCR-NK cells for use in a method of treatment, wherein the population has been generated by a method according to any one of claims 1 to 48.

53. A population comprising TCR-NK cells for use in a method of treatment, wherein, prior to administration, the TCR-NK cells are contacted in vitro with an anti-CD3 agent.
